SEQUENCE LISTING (17 sequences)

SEQ ID NO: 1 - DNA, 629 bp, Arabidopsis thaliana
  misc_feature 455, 464, 467, 475, 497, 500, 508, 514, 519, 536, 543,544, 576, 583, 584, 597: n = A,T,C or G
tgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactgggagaatgtggaatatgcctgtccataaatgggatgggtccgacatatataccttccccgtgcttgcgcacaaggattacccaaagacaccccggccattaaccattggctttcccaagccccctggaggcctttccatgggccanggacccggngtnccctggcnggcccttcaaagcaaagggggnttncctggggntaaagntccangggcccttggggcccanccaaaannttcccccgggaaagggttgcccaccggggggngaaaaanncccgggggcaccncggaattttgggaacccggggggggccttttt

SEQ ID NO: 2 - DNA, 1904 bp, Arabidopsis thaliana
  CDS (139)...(1701)
atttcttagcttcttccttcaatccgctctttccctctccattagattctgtttcctctttcaatttcttctgcatgcttctcgattctctctgacgcctcttttctcccgacgctgtttcgtcaaacgcttttcgaaatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgaaacaactgttcaaaaaatgactttcttcaaacatctatggcctcgttggatctccgttgatgttgtggtggttctgatgctaaaacgacaaatagtgttataaccattgaagaagaaaagaaaattagagttgttgtatctgcaaaaattttggtagagacacgcaaacccgtttggattttgttatggtgtaaagcggccgc

SEQ ID NO: 3 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 4 - DNA, 1537 bp, Brassica napus
  CDS (2)...(1107)
ttaaaacctaactctttgcaaatggagattttggattctggaggcgtcactatgccgacggagaacggtggtgccgatctcgatacgcttcgtcaccggaaaccgagatcggattcttccaatggacttcttcctgattccgtaactgtttccgatgctgacgtgagggatcgggttgattcagctgttgaggatactcaaggaaaagccaatttggccggagaaaacgaaattagggaatccggtggagaagcggggggaaacgtggatgtaaggtacacgtatcggccgtcggttccagctcatcggagggtgcgggagagtccactcagctctgacgccatcttcaaacagagccatgctggactattcaacctgtgtgtagtagttcttgttgctgtaaacagtagactcatcatcgaaaatctcatgaagtacggttggttgatcagaactgatttctggtttagttcaacgtctctgcgagattggccccttttcatgtgttgtctctccctttcaatctttcctttggctgcctttaccgtcgagaaattagtacttcagaaatgcatatctgaacctgttgtcatcattcttcatattattatcaccatgaccgaggtcttgtatccagtctatgtcactctaaggtgtgattccgccttcttatcaggtgtcacgttgatgctcctcacttgcattgtgtggctgaagttggtttcttacgctcatactaactatgacataagaaccctagctaattcatctgataaggccaatcctgaagtctcctactatgttagcttgaagagcttggcgtatttcatgcttgctcccacattgtgttatcagccgagctatccacgttctccatgtatccggaagggttgggtggctcgtcaatttgcaaagctgatcatattcactggattcatgggatttataatagagcaatatataaatcctattgttaggaactcaaaacatcctttgaaaggggatctcttatacggtgttgaaagagtgttgaagctttcagttccaaatttatacgtgtggctctgcatgttctactgcttcttccacctttggttaaacatattggcagagctcctctgcttcggggatcgtgaattctacaaagattggtggaatgcaaaaagcgtgggagattattggagaatgtggaatatgcctgttcataaatggatggttcgacatgtatactttccgtgccttcgcagaaatataccgaaagtacccgctattatccttgctttcttagtctctgcagtctttcatgagttatgcatcgcagttccttgtcgtctcttcaaactatgggctttcttggggattatgtttcaggtgcctttggtatttatcacaaactacctacaagaaaggtttggctccatggtgggaaacatgatattctggtttaccttctgcattttcggacaaccgatgtgtgtgcttctttattatcacgacttgatgaaccgcaaaggaaagatgtcatagatgtgtatgt

SEQ ID NO: 5 - protein, 344 aa, Brassica napus
NLTLCKWRFWILEASLCRRRTVVPISIRFVTGNRDRILPMDFFLIPLFPMLTGIGLIQLLRILKEKPIWPEKTKLGNPVEKRGETWMGTRIGRRFQLIGGCGRVHSALTPSSNRAMLDYSTCVFLLLTVDSSSKISSTVGSELISGLVQRLCEIGPFSCVVSPFQSFLWLPLPSRNYFRNAYLNLLSSFFILLSPPRSCIQSMSLGVIPPSYQVSRCSSLALCGSWFLTLILTMTEPLIHLIRPILKSPTMLARAWRISCLLPHCVISRAIHVLHVSGRVGWLVNLQSSYSLDSWDLSNIILLLGTQNILKGISYTVLKECSFQFQIYTCGSACSTASSTFGTY

SEQ ID NO: 6 - DNA, 1446 bp, Brassica napus
  CDS (82)...(1107)
cgaaaatctcatgaagtacggttggttgatcagaactgatttctggtttagttcaacgtcgctgcgagattgccgcttttcatgtgttgtctctccctttcaatctttcctttggctgcctttaccgtcgagaaattagtacttcagaaatgcatatctgaacctgttgtcatctttcttcatgttattatcaccatgaccgaggtcttgtatccagtctatgtcactctaaggtgtgattctgccttcttatcaggtgacacgttgatgctcctcacttgcattgtgtggctgaagttggtttcttacgctcatactaactatgacataagaaccctagctaattcatctgataaggccaatcctgaagtctcctactatgttagcttgaagagcttggcttatttcatgcttgctcccacattgtgttatcagccaagctatccacgttctccatgtatccggaagggttgggtggctcgtcaatttgcaaaactggtcatattcactggactcatgggatttataatagagcaatatataaatcctattgttaggaactcaaagcatcctctgaaaggggaccttctatatgctattgaaagagtgttgaagctttcagttccaaatctatatgtgtggctctgcatgttctactgcttcttccacctttggttaaacatattggcagagctcctctgcttcggggaccgtgaattctacaaagattggtggaatgcaaaaagcgttggagattattggagaatgtggaatatgcctgttcacaaatggatggttcgacatgtatactttccgtgcctgcgcatcaagataccaaaagtacccgccattatcattgctttcttagtctctgcagtctttcatgagttatgcatcgcagttccttgccgtctcttcaatctatgggctttcatgggaattatgtttcaggtccctttggtctttatcacaaactttttacaagaaaggtttggctccatggtgggaaacatgatctttggttcagcttcttgcattttcggacaaccgatgtgtgggcttctttattaccatgacctgatgaaccgcaaaggatccatgtcctgaaaaggactttttacgccccaaaaaaaaaattggtcaattggaaaaatgggagtttttgtatccttttggtagccgttaaaatgcctttaaaaagacgaatcctttggagttcttgtttctcttggtctctgtcccccacgggattttctatttctcgtcttttaacaagcccataaaaaaaagtagactgagataattggattttgttatgctgtaaaaaaaatttcattcaaaaatgtttgaataatctttgacgattcccaaaatcccgagaaaaataaaagtaagcctttcctttttaaaaaaaaaaaaaaaaaaaaaaaaaaaaaaaaaaaaaaa

SEQ ID NO: 7 - protein, 341 aa, Brassica napus
MCCLSLSIFPLAAFTVEKLVLQKCISEPVVIFLHVIITMTEVLYPVYVTLRCDSAFLSGDTLMLLTCIVWLKLVSYAHTNYDIRTLANSSDKANPEVSYYVSLKSLAYFMLAPTLCYQPSYPRSPCIRKGWVARQFAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFGSASCIFGQPMCGLLYYHDLMNRKGSMS

SEQ ID NO: 8 - DNA, 1512 bp, Brassica napus
  CDS (1)...(1512)
atggcgattttggattctggaggcgtcgctgtaccgccgacggagaacggcgtcgcggatctcgacaggctccaccgtcgtaaatcgagttcggattcttccaacggactcctctccgatacttccccgtcggacgatgttggagctgcggcggccgaaagggatcgggttgattccgctgccgaggaggaggctcagggaacagcgaatttagctggcggagatgccgaaactagggaatccgccggaggcgatgtaaggtttacgtatcgaccgtcggttccagctcatcggaggacgagggagagtcctctcagctccgacgctatcttcaaacaaagccatgcaggattgttcaacctctgtgtagttgttcttgttgctgttaacagtagactcatcatcgaaaacctcatgaagtatggttggttgatcagaactgatttttggtttagttctacatccttacgagactggccgcttttcatgtgttgtctttcactttcggtctttcctttggctgccttcacggtcgagaaaatggtacttcagaaattcatatctgagcctgttgccatcattcttcatgtcattataaccatgacagaggtcttgtatccagtctacgtcacactgaggtgtgattctgccttcttgtcaggtgtcacgttgatgctgctcacttgcattgtgtggctgaagttggtttcttacgctcatactagctacgacataagaaccctggccaattcagctgataaggtcgatcctgaaatctcctactatgttagcttgaagagcttggcgtatttcatggttgctcccacactgtgttatcagccaagctatccacgttctccatgtatccggaagggttgggtggctcgtcaacttgcaaaactggtcatattcactggactcatgggatttataatagagcaatatataaatcctattgttaggaactcaaagcatcctctgaaaggggaccttctatatgctattgaaagagtgttgaagctttcagttccaaatctatatgtgtggctctgcatgttctactgcttcttccacctttggttaaacatattggcagagctcctctgcttcggggaccgtgaattctacaaagattggtggaatgcaaaaagcgttggagattattggagaatgtggaatatgcctgttcacaaatggatggttcgacatgtatactttccgtgcctgcgcatcaagataccaaaagtacccgccattatcattgctttcttagtctctgcagtctttcatgagttatgcatcgcagttccttgccgtctcttcaatctatgggctttcatgggaattatgtttcaggtccctttggtctttatcacaaactttttacaagaaaggtttggctccatggtgggaaacatgatctttggttcagcttcttgcattttcggacaaccgatgtgtgggcttctttattaccatgacctgatgaaccgcaaaggatccatgtcctga

SEQ ID NO: 9 - protein, 503 aa, Brassica napus
MAILDSGGVAVPPTENGVADLDRLHRRKSSSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRLIIENLMKYGWLIRTDFWFSSTSLRDWPLFMCCLSLSVFPLAAFTVEKMVLQKFISEPVAIILHVIITMTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQLAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFGSASCIFGQPMCGLLYYHDLMNRKGSMS

SEQ ID NO: 10 - DNA, 2090 bp, Tropaeolum majus
  CDS (171)...(1727)
acgcggggagttttcaaaatcatattatgctttttcttcactactgcatgaactttctttctacttcttgcaactgatttgtaatccttacacatgtttctagttttctccatataaaaaaaatattctctgagcttctcgattctctagagagagaaggccaaaaaaaaatggcggtggcagagtcgtcacagaacacgacaaccatgagtggtcacggcgactcggatctcaacaatttccgtagaaggaaaccgagttcctccgtgattgaaccttcgtcgtccggttttacatccaccaatggcgtaccggcgactggccacgtggctgagaatcgtgaccaggatcgggtaggggctatggagaacgcaacaggatcggtcaacttaattggaaatggtggaggcgtggttatcgggaatgaagagaaacaggtaggggagactgatatacgattcacttaccggccttcgtttccggctcatcggagggtgagggagagtcctcttagctctgatgcaatcttcaaacagagccatgcgggtttattcaacttgtgtatagtagtgctcattgcagtaaacagtaggcttatcatcgaaaatcttatgaagtatggttggttgatcgatactggtttctggtttaactcaagatcactgggtgattggtccatctttatgtgctgtcttacactcccaattttcccacttgctgcttttattgttgaaaagctggtgcagcgaaatcatatatctgaacttgttgctgttctccttcatgtaatcgtttctaccgctgcagttttatatccagttattgtgatcttaacgtgtgattcggtgtatatgtctggtgtggtattgatgctctttggttgcattatgtggttgaagctggtgtcatatgcacatactagttctgatattagaacactggccaaatctggctataagggggatgcgcaccccaattcaaccattgtgagttgctcatatgatgttagcttgaagagtttggcatacttcatggtggcgccgacattatgttaccagcctagctatcctcgttcgtcgtgtatccgcaagggttgggttgttcgtcaatttgtcaaactaatagttttcataggactcatggggttcattatagaacaatatattaatcctatcgttcgaaattccaaacacccattgaaaggagattttttatatgcaatagaaagagttttgaagctttcagttccaaatctatatgtttggctttgcatgttctactcttttttccacctctggttgaacatactggctgagcttcttcgctttggtgatcgtgaattctacaaagattggtggaatgcaaaaactgttgcggagtattggaaaatgtggaatatgcctgttcatagatggatggttcgtcatctatattttccctgtttgaggaatgggatacccaaggaaggtgccattattatcgcgttcttagtttctggtgctttccatgagctctgcattgcagttccttgccacgtattcaagttatgggcctttataggcattatgtttcaggttcccttggtattgattacgaattatctacaagaaaagttcagtaattctatggtgggcaatatgatcttctggttcatcttctgcatacttggccaacctatgtgtgtccttctatattaccatgacctgataaatctaaaggaaaagtgaaaaaatggaagttgcctatgctcagagtattcctatcccaatgcacacattatatggttctgtacaatctgtgcccccttcatcctttacacgtacccatgctggttcctgcacgatgatttgccttttgtttgtaagcaatatttggagagagtccaatttaggaagtgactagtgtggcttatatcttgtatactacctttagtcatgggggggtttttatattactagtaccaaaagtcaagttgtatatgatttacggtttagtttctttcatgttttttgtttttgtgtaaatatacgtttcatatatcactgttttttcaaagtaaaatcaataataccccatagatgttgaaactg

SEQ ID NO: 11 - protein, 518 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFNSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 12 - DNA, 2099 bp, Nicotiana tabacum
  CDS (208)...(1806)
ggcacgagcgaaatcttacccaatcctccgttgcttttcttttagatcctctttttctgtcattctctttttcccaataacaacaactcattgcatgtgaaggttgattttgatttttgtgtttattcaaactctctcttcacgattttcttactctttctagaagtatccattactttttagtctgtgattcggcgaaagtaagcaatggtgatcatggaattgccggagagcgtcgaaatgacgacgacgacgacgacttcgggtattgagaacctcaactccgatcttaatcactcggttcggaggagacgtggcagtaatggttttgaggcggctagtgcaattaacagttcggatgcgaatatgagcgaagatagaagagatgtgtgtggcagcggtgctggattggaaacggtgaatgagcggagtaaatcggttggtgagtccagtgatgtaattcgaaaggaggacgacaggaatgataatgttgcgaatggtgaggaaagcaaatcaacggaaacaacaacgacgccgtttaaatttgcttacagggcgtcggcaccagctcaccggcgaatcaaggagagtcctctcagctccgacgccattttcaaacagagtcacgcaggcctgttcaatctctgtgtggtggtgctgattgctgttaacagcaggctgattatcgagaacttgatgaagtatggccttttaattagggctggcttttggtttagctcgaagtcgttgagggattggccgcttctaatgtgctgtctcagtctccaaattttgccgctcgctgcttttcttgtggagaagttggcacagcagaggcatttgactgagcgtgcggtggttactcttcacataactataacaacagctgccattttgtatccagttctggtcattcttgggtgtgattctgcttttctgtttggtgtcatattgatgctggttgcttgcattgtgtggatgaagctggtttcttacgcacatacaaatcatgatatgagacagctcgcaaagtctacggacaaggatgaaacttcagatggggatttctcttatgatgttagcttcaagagtttggcttacttcatggttgcgccaacattatgttatcagcttagctatccccacactccatgcattcgcaaaggttgggtggcacgccaattcatcaagctggtaatatttacaggattgatgggatttatcatagaacagtacattaacccaattgtgcaaaactcacaacatcctttgaaaggaaaccttttatatgccatcgagagggtattgaagctttcggttccaaatttatatgtctggctctgcatgttttactgcttctttcatctttggctaaatatacttgcggaactactatgttttggtgatcgtgagttctacaaggattggtggaatgccaaaacaattgatgagtactggaggatgtggaatatgcctgttcataagtggatggttcgtcacatttatttcccttgcttaagaaacggaattccaaagggggtcgcaatactgattgctttccttgtatctgctgttttccacgagctgtgtattgctgttccatgtcgccttttcaagtggtgggcattcatgggaattatgttccaggttcctttggtcatactcacaaacttcttacaaaacaagttccaaagctcgatggtgggcaatatgatgttctggtgctttttctgcattcttggtcagccaatgtgtgtgcttctgtattaccacgatgtgatgaatagaaaaagcagtgcacgttaagcttcatccagggatgaattgttgtatgagcaagtattttaagttttggatcccaagctctattctactgtttctggcaaggcattcctgctatttccttcatcagttccaacaatattcagatgatacgaaatatctgtttggaatgcacaacacaagccacggccagagatgctgatgtctcacattttattgtgttcttcatgtcggagaaatgtaaaatactatcttgagataactctcatgttagtaaatacctttttgcctctaaaaaaaaaaaaaaaaaaaaaaaa

SEQ ID NO: 13 - protein, 532 aa, Nicotiana tabacum
MVIMELPESVEMTTTTTTSGIENLNSDLNHSVRRRRGSNGFEAASAINSSDANMSEDRRDVCGSGAGLETVNERSKSVGESSDVIRKEDDRNDNVANGEESKSTETTTTPFKFAYRASAPAHRRIKESPLSSDAIFKQSHAGLFNLCVVVLIAVNSRLIIENLMKYGLLIRAGFWFSSKSLRDWPLLMCCLSLQILPLAAFLVEKLAQQRHLTERAVVTLHITITTAAILYPVLVILGCDSAFLFGVILMLVACIVWMKLVSYAHTNHDMRQLAKSTDKDETSDGDFSYDVSFKSLAYFMVAPTLCYQLSYPHTPCIRKGWVARQFIKLVIFTGLMGFIIEQYINPIVQNSQHPLKGNLLYAIERVLKLSVPNLYVWLCMFYCFFHLWLNILAELLCFGDREFYKDWWNAKTIDEYWRMWNMPVHKWMVRHIYFPCLRNGIPKGVAILIAFLVSAVFHELCIAVPCRLFKWWAFMGIMFQVPLVILTNFLQNKFQSSMVGNMMFWCFFCILGQPMCVLLYYHDVMNRKSSAR

SEQ ID NO: 14 - DNA, 1964 bp, Piralla frutescens
  CDS (69)...(1673)
tttagaaccaaactattctccgttaagttctgagttcgatttctttcttttctcaaattttccgtgcgatggcgatcttggactcgccggagatcctggacacgacgtcgtccagtgccgacaacggcgccgcacatcacaccactcttcgccggagacaaagtgcgcgctccgttccgcctcttctcgactccgattccaactctctggaggcagagagcgcaatcaatgattccgaaaatgttcgaaacgacgctaatttgatcgaaaatctccgcggcggagccgtggaatccgagaacgaaaaacaggagagttatggtaaggaggagggggcgaaagtgaaggagaatggagaaactagtaatggcaacggaactgatgttatggccgtcaaattcacattcaggccggcggcgcctgctcaccgcaaaaataaggagagtcctcttagctccgacgccatcttcaaacagagccatgcaggcctcttcaacctttgtatagtggtgcttgttgctgtaaatagcagactaataattgagaatttaatgaagtatgggtggctgatcaaatcaggattttggtttagttcaacatcgcttagggattggccactgctaatgtgttgtcttagtcttccagtttttgcactcgcttcatttcttgtcgagaagttggtgaaactaaattatatacctgagtgggtcgcagtctttcttcatgttacaatcacaacagtggaaatcttgtttccagttgttgtcattcttaggtgtgattctgctgttctatcaggtgtcacgctaatgctctttgcttgcactgtatggttgaagctcgtttcctacgcacatacaaactatgatttgagagtacttgcaaaatcacttgataagtgggaagctatgtccaggtactggaacctcgactacgcttatgatgtaagctttaagagtctggcatacttcatggttgctcctacattgtgttaccagccaagctaccctcggacagcttgcattcggaagggttgggtggtaaggcaactaattaagctggtaatattcacaggactcatgggatttattatagaacagtacataaacccgatcgttcaaaattctcaacatcctctgaaaggaaaccttttatatgccattgagagggtcttgaagctttctgttccaaatttatatgtgtggctctgcatgttttattgttttttccacctctggctaaatatacttgctgaacttctgtgctttggggaccgtgagttttataaggattggtggaatgcgaggacagtggaggagtactggagaatgtggaatatgcctgtccataaatggatggttcggcatatatactgtccatgcttacaaaatggaataccaaagatagtggcagttttgatcgcgtttcttgtgtctgcgatttttcatgagctgtgcgttgcagtcccttgccaaatattcaagttttgggcgttctcgggtatcatgcttcaggttcctctcgtaatcgtgactaattacttgcaagaaaagttcaaaaactcaatggtgggcaatatgatgttctggtgcttcttctgtatctttggtcaacctatgtgtgtgttgctgtactaccacgacttgatgaatcgaaaagctagtgcaaggtagggatgtgattcatcttctgagtagaaatctaaagctcaccagccccaacccacccgaaaaacaaaaaggagcaaggatcctgattgtgagctggtagataatttgctacaactatgtttcttaaatagctgggagtagtttgttatctgccttcacctaggacgacgttatgatctgttgtgatgggggtaagggggcatgcaaattttgtctatttttcaaggaatacagaaatggtgaaaatttgatgaagcatacccctcgtttactgacaaaaaaaaaaaaaaaaaa

SEQ ID NO: 15 - protein, 534 aa, Piralla frutescens
MAILDSPEILDTTSSSADNGAAHHTTLRRRQSARSVPPLLDSDSNSLEAESAINDSENVRNDANLIENLRGGAVESENEKQESYGKEEGAKVKENGETSNGNGTDVMAVKFTFRPAAPAHRKNKESPLSSDAIFKQSHAGLFNLCIVVLVAVNSRLIIENLMKYGWLIKSGFWFSSTSLRDWPLLMCCLSLPVFALASFLVEKLVKLNYIPEWVAVFLHVTITTVEILFPVVVILRCDSAVLSGVTLMLFACTVWLKLVSYAHTNYDLRVLAKSLDKWEAMSRYWNLDYAYDVSFKSLAYFMVAPTLCYQPSYPRTACIRKGWVVRQLIKLVIFTGLMGFIIEQYINPIVQNSQHPLKGNLLYAIERVLKLSVPNLYVWLCMFYCFFHLWLNILAELLCFGDREFYKDWWNARTVEEYWRMWNMPVHKWMVRHIYCPCLQNGIPKIVAVLIAFLVSAIFHELCVAVPCQIFKFWAFSGIMLQVPLVIVTNYLQEKFKNSMVGNMMFWCFFCIFGQPMCVLLYYHDLMNRKASAR

SEQ ID NO: 16 - DNA, 1181 bp, Zea mays
  misc_feature 235, 236, 237, 238, 239, 317, 318, 319, 320, 321, 322,393, 394, 395, 396, 397, 398: n = A,T,C or G
ccacgcgtccggtctcttatgcacatacaaattatgatataagggtattgtccaaaagtactgagaagggtgctgcatatggaaattatgtcgatcctgagaatatgaaagatccaacctttaaaagtctagtgtacttcatgttggccccaacactttgttaccagccaacttatcctcaaactacatgtattagaaagggttgggtgacccagcaactcataaagtgcgtggnnnnnacaggcttgatgggcttcataattgagcaatatataaacccaattgtgaagaattccaaacatccactgaaagggaannnnnngaatgctatagaaagagtcttaaaactctcagtgccaacattatatgtatggctttgcatgttctattgcnnnnnncatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtattccatgagatatgtattgcggtgccgtgccacattttcaaattctgggcattttctgggatcatgtttcagataccgttggtattcttgacaagatatctccatgctacgttcaagcatgtaatggtgggcaacatgatattttggttcttcagtatagtcggacagccgatgtgtgtccttctatactaccatgacgtcatgaacaggcaggcccaggcaagtagatagttcggcagagacatgtacttcaacatcgaccatcagaagcagactggagcgacgcggcaggaagcagcagtagcccctcacttgccattgtttaccatcagctagcagctggtcagcctgaaaatgcttctattccaggcggccgtgctgggatgcagcgtgtacattaccagcgatgcaacgtcaacggttaccaagcgcggataagcagtgctcgtggcatgtgaactctgtacacattgaagtctatatgtccgcggtgctgtgtgcataatgtcgtggtagatgtaatatgtacaatgtatgccacgacttaagaggtcaaaatgaggctcacgt

SEQ ID NO: 17 - DNA, 1572 bp, Zea mays
ttttggtttaatgctacatcattgcgagactggccactgctaatgtgttgccttagtctacccatatttccccttggtgcatttgcagtcgaaaagttggcattcaacaatctcgttagtgatcctgctactacctgttttcacatcctttttacaacatttgaaattgtatatccagtgctcgtgattcttaagtgtgattctgcagttttatcaggctttgtgttgatgtttattgcctgcattgtttggctgaagcttgtatcttttgcacatacaaaccatgatataagaaaactgatcacaagcggcaagaaggttgataatgaactgaccgcggctggcatagataatttacaagctccaactcttgggagtctaacatacttcatgatggctccgacactctgttatcagccaagttatcctcgaacaccttatgttagaaaaggttggctggtccgtcaagttattctatacttgatatttactggtctccaaggattcattattgagcaatacataaatcctattgttgtgaactctcaacatccattgatgggaggattactgaatgctgtagagactgttttgaagctctcattaccaaatgtctacctgtggctttgcatgttttattgccttttccatctgtggttaaacatacttgctgagattcttcgatttggtgaccgagaattctacaaagactggtggaatgcaaagacaattgatgagtactggagaaaatggaacatgcctgtgcataaatggattgttcgtcatatatattttccttgcatgcgaaatggtatatcaaaggaagttgctgtttttatatcgttctttgtttctgctgtacttcatgaggtaacttatttactttttcactcttcatctgcatatattaattatatagttctctattttcaaatgtgtcctttcgagtttcgacatgcttttgttcaaacttaccagctgtagattacttggatgaagtgctctatataaaattcaatatttcacaatccagtccctttcgagaaaattatgatacattttgtttgcatttgtacaccagttatgcgttgcagttccctgccacatactcaagttctgggctttcttaggaatcatgcttcagattcccctcatcatattgacatcatacctcaaaaataaattcagtgacacaatggttggcaatatgatcttttggttttttttctgcatatacgggcagccaatgtgtgttctattgtattaccatgatgtgatgaaccggactgagaaggcaaaataaccatctgtagatcttttttggtgtttcatttctgccatcatggaaactgaaagcaataatctgtgcacacagtaaaccagcatcgtgtcttccagttttctttttgttgttggaatctatcctagatctttatcatgtgtatggtggataacctcatgtcaccatcgtatctgtatacaataagcctaaatcagctgacgttatatatgtaaattagtaaatgtaatgactaattagtgccaagaaaaaaaaaaaaaaaaaaaaaaaaa

(19) United States
(12) Patent Application Publication
(10) Pub. No.: US 2003/0074695 A1
(43) Pub. Date: Apr. 17, 2003

(54) Plant diacylglycerol O-acyltransferase and uses thereof

(76) Inventors: Robert V. Farese JR., San Francisco, CA (US); Sylvaine Cases, Belmont, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/223,076

(22) Filed: Aug. 15, 2002

Related U.S. Application Data
(63) Continuation-in-part of application No. 10/040,315, filed on Oct. 29, 2001.
Application No. 10/040,315 is a continuation-in-part of application No. 09/339,472, filed on Jun. 23, 1999, now abandoned.
Application No. 10/040,315 is a continuation-in-part of application No. PCT/US98/17883, filed on Aug. 28, 1998.
Application No. PCT/US98/17883 is a continuation-in-part of application No. 09/103,754, filed on Jun. 24, 1998, now Pat. No. 6,344,548.
(60) Provisional application No. 60/107,771, filed on Nov. 9, 1998.

(51) Int. Cl. A01H 5/00; C07H 21/04; C12N 9/06; C12N 9/10; C12N 15/82; C12N 5/04

(52) U.S. Cl. 800/281; 435/69.1; 435/191; 435/193; 435/320.1; 435/419; 536/23.2

(57) ABSTRACT

Plant nucleic acid compositions encoding polypeptide products with diacylglyceride acyltransferase (DGAT) activity, as well as the polypeptide products encoded thereby and methods for producing the same, are provided. Methods and compositions for modulating DGAT activity in a plant, particularly DGAT activity in plant seeds, and transgenic plants with altered DGAT activity are provided. Such plants and seeds are useful in the production of human food and animal feedstuff, and have several other industrial applications. Also provided are methods for making triglycerides and triglyceride compositions, as well as the compositions produced by these methods. The subject methods and compositions find use in a variety of different applications, including research, medicine, agriculture and industry.

### CROSS REFERENCE TO RELATED APPLICATIONS

This application is a continuation-in-part of application Ser. No. 10/040,315 filed Oct. 29, 2001; which application is: (a) a continuation-in-part of application Ser. No. 09/339,472 filed on Jun. 23, 1999, which application claims priority to the filing date of United States Provisional Patent Application Serial No. 60/107,771 filed Nov. 9, 1998; and (b) a continuation-in-part of PCT application serial no. PCT/US98/17883, filed Aug. 28, 1998, which application is a continuation in part of application Ser. No. 09/103,754, now U.S. Pat. No. 6,344,548, filed Jun. 24, 1998; the disclosures of which applications are herein incorporated by reference.

### ACKNOWLEDGMENT OF GOVERNMENT SUPPORT

This invention was made with Government support under Grant Nos. R01 52069 and R01 57170 awarded by the National Institute of health and support from the Veterans Administration. The Government has certain rights in this invention.

### INTRODUCTION

1. Field of the Invention

The field of the invention is plant enzymes, particularly plant acyltransferases.

2. Background of the Invention

Triacylglycerol is synthesized by the sequential transfer of acyl chains to a glycerol backbone by a series of enzymes in the Kennedy pathway. These enzymes are glycerol-3-phosphate acyltransferase (which adds a first acyl chain to a glycerol backbone to form a glycerol-3-phosphate), lysophosphatidic acid acyltransferase (which adds a second acyl chain to glycerol-3-phosphate to form diacylglycerol) and diacylglycerol acyltransferase (DGAT), which transfers a third acyl chain to diacylglycerol to form triacylglycerol (TAG) (see Topfer el al. Science 1995 268: 681-686).

Because of its high carbon content, TAG is an important molecule for storage of energy in plants, particularly in seeds where it is used as an energy source for germination. Because of its unique properties, TAG is a valuable component of animal feedstuffs and a major component of the purified vegetable oils consumed by man. In addition, many TAGs have other uses, including as a “bio-fuel”, in industrial lubricants, surfactants, paints and varnishes, and in various soaps and cosmetics. The ability to manipulate the biosynthesis of TAG using DGAT is a major goal of plant biotechnology.

Because the reaction catalyzed by DGAT is unique to the Kennedy pathway, the reaction is thought to be at a critical branchpoint in glycerolipid biosynthesis. Enzymes at such branchpoints are considered prime candidates for sites of metabolic engineering, and, as such, DGAT has become an enzyme of intense interest because of its potential as a regulator of TAG biosynthesis. Furthermore, several biochemical studies indicate that TAG enzymes from different plant species are specific for certain acyl chains, suggesting that the plant DGAT could be used to manipulate the levels of saturated fatty acids in an oil or acyl chain length in TAG, in addition to the absolute levels of TAG biosynthesis.

Because of its central role in the regulation of TAG biosynthesis in plants, there is much interest in the identification of plant DGAT polypeptides and polynucleotides that encode them. Previous attempts to identify plant DGAT polypeptides have, in general, failed, primarily because the DGAT enzyme is difficult to purify. As such, a great need still exists for plant DGAT proteins and polynucleotides that encode them. The present invention addresses these, and other, needs.

Relevant Literature

References of interest include: U.S. Pat. No. 6,100,077; Cases et al., the FASEB Journal (Mar. 20, 1998) Vol. 12., No.(5):A814; Cases et al., Proc. Natl. Acad. Sci. USA (October 1998) 95:13018-13023; Garver et al., Analytical Biochemistry (1992) 207: 335-340; Shockey et al., Plant Phys. (1995) 107: 155-160; Little et al Biochem. J. (1994) 304: 951-958; Kamisaki at al., J. Biochem (1994) 116:1295-1301; Katavic et al., Plant Phys. (1995) 108:399-409 Zou et al., Plant Mol. Bio. (1996) 31:429-433; Kamisaki et al J. Biochem (1996) 119: 520-523; Oelkers et al J. Bio. Chem. (1988) 41: 26765-26771; Kamisaki et al., J. Biochem (1997) 121: 1107-1114; Lassner et al., The Plant Cell (1996) 8: 281-292) and Genbank Accession No.AF078752 (Nov. 11, 1998); Genbank Accession No. AAC63997 (Oct. 15, 1998); and Genbank Accession No. AF059202 (Oct. 15, 1998).

### SUMMARY OF THE INVENTION

Plant nucleic acid compositions encoding polypeptide products with diacylglyceride acyltransferase (DGAT) activity, as well as the polypeptide products encoded thereby and methods for producing the same, are provided. Methods and compositions for modulating DGAT activity in a plant, particularly DGAT activity in plant seeds, and transgenic plants with altered DGAT activity are provided. Such plants and seeds are useful in the production of human food and animal feedstuff, and have several other industrial applications. Also provided are methods for making triglycerides and triglyceride compositions, as well as the compositions produced by these methods. The subject methods and compositions find use in a variety of different applications, including research, medicine, agriculture and industry.

### BRIEF DESCRIPTION OF THE FIGURES

FIG. 1 shows Arabidopsis thaliana Genbank accession number AA042298 (SEQ ID NO:1)

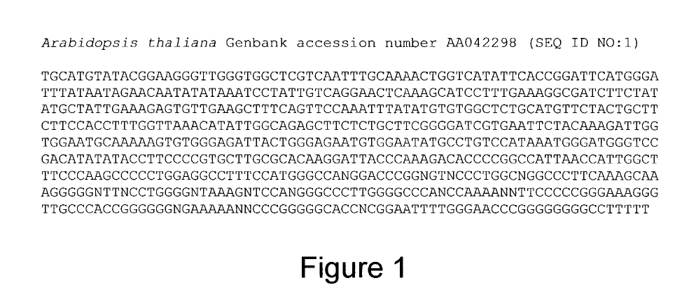

FIG. 2 shows Arabidopsis thaliana Genbank accession number ATH238008 (SEQ ID NO:2)

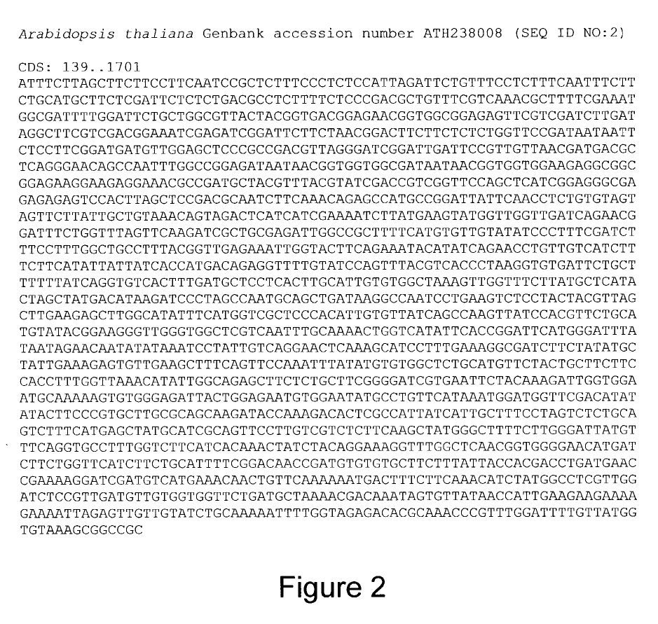

FIG. 3 shows Arabidopsis thaliana Genbank accession number CAB45373 (SEQ ID NO:3)

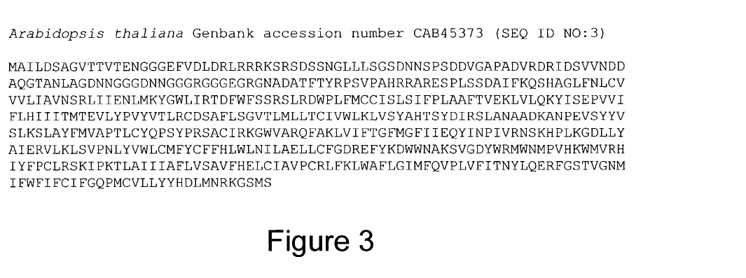

FIG. 4 shows Brassica napus Genbank accession number AF251794 (SEQ ID NO:4)

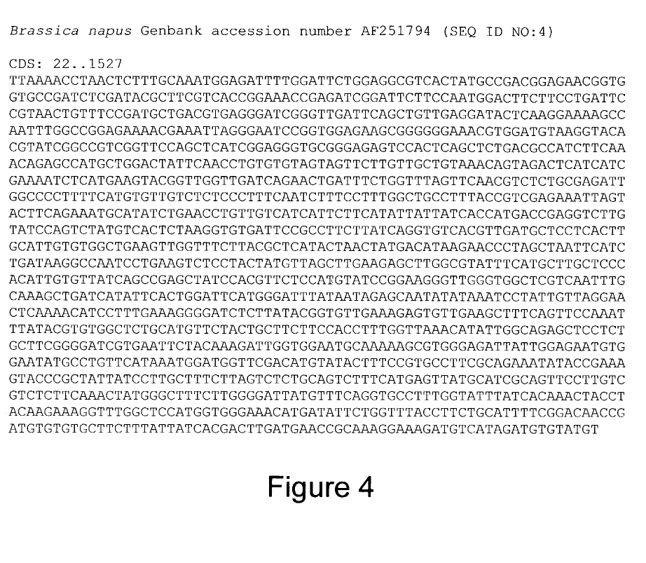

FIG. 5 shows Brassica napus Genbank accession number AAF64065 (SEQ ID NO:5)

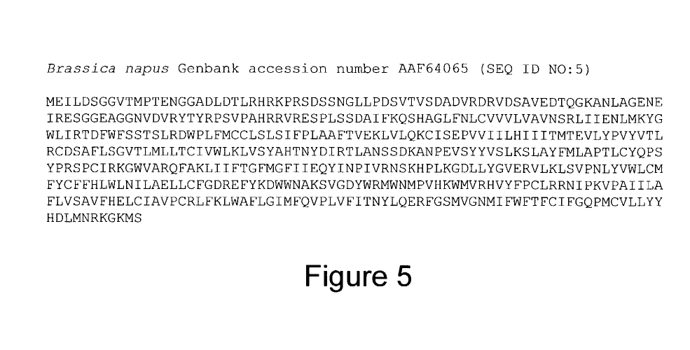

FIG. 6 shows Brassica napus Genbank accession number AF155224 (SEQ ID NO:6)

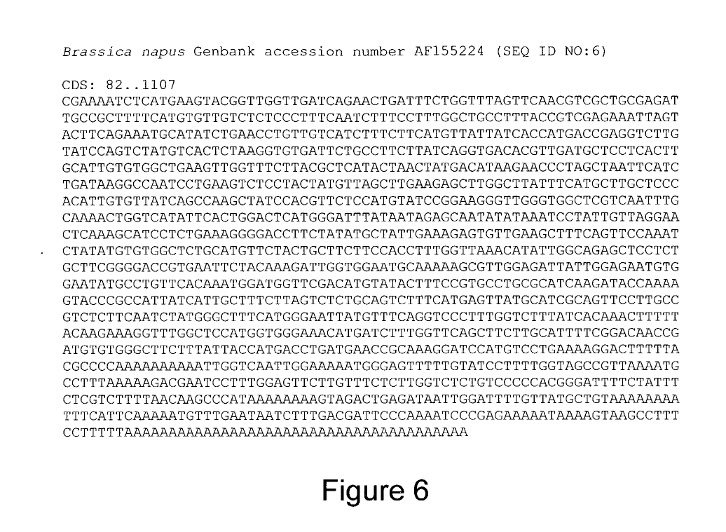

FIG. 7 shows Brassica napus Genbank accession number AAD40881 (SEQ ID NO:7)

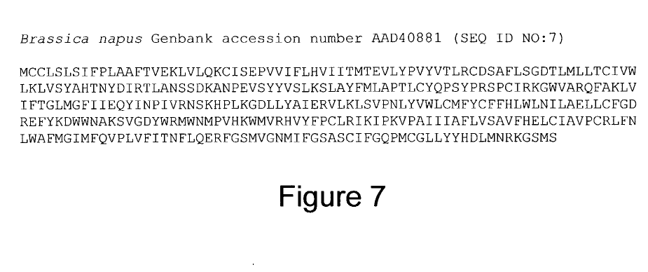

FIG. 8 shows Brassica napus Genbank accession number AF164434 (SEQ ID NO:8)

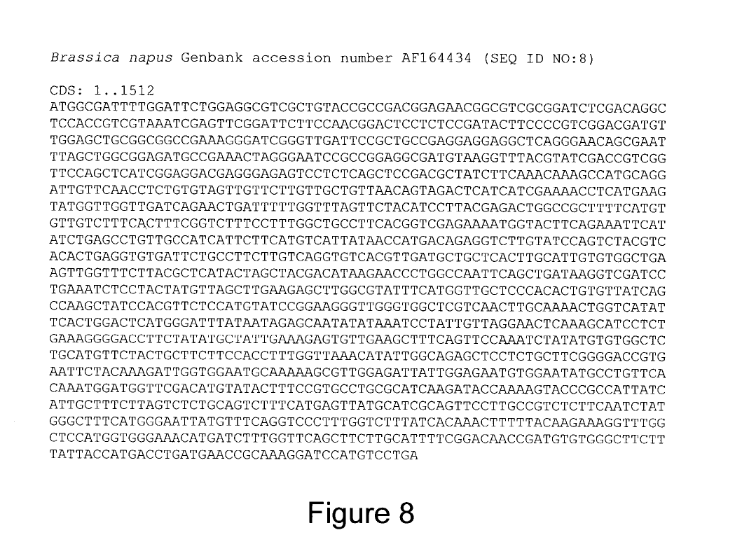

FIG. 9 shows Brassica napus Genbank accession number AAD45536 (SEQ ID NO:9)

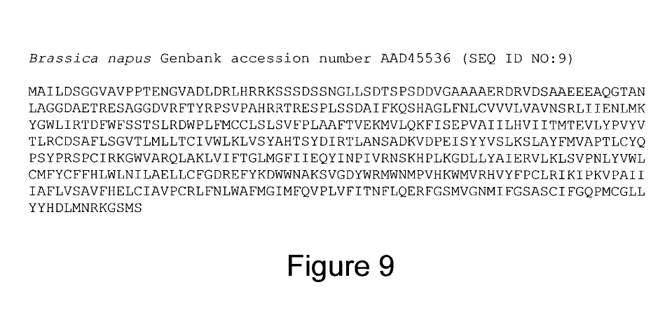

FIG. 10 shows Tropaeolum majus Genbank accession number AY084052 (SEQ ID NO:10)

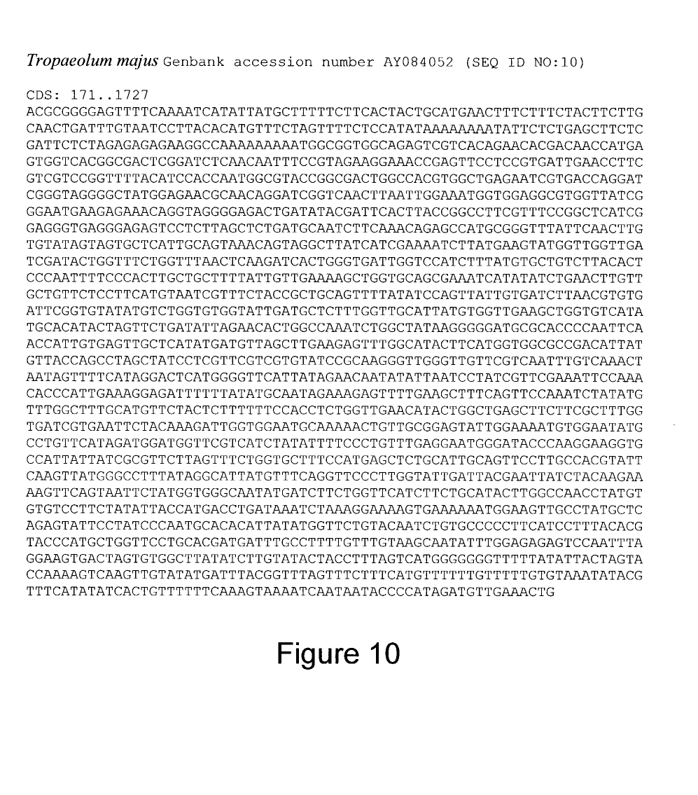

FIG. 11 shows Tropaeolum majus Genbank accession number AAM03340 (SEQ ID NO:11)

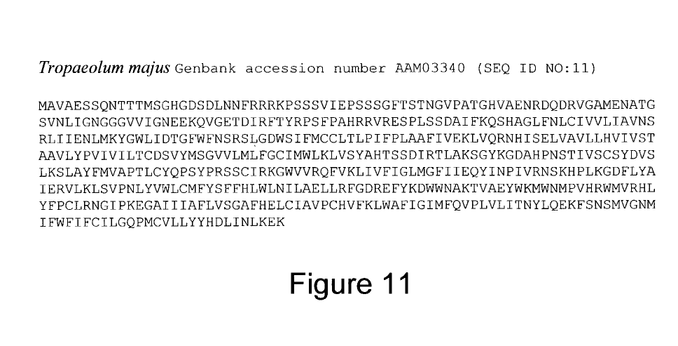

FIG. 12 shows Nicotiana tabacum Genbank accession number AF1 29003 (SEQ ID NO:12)

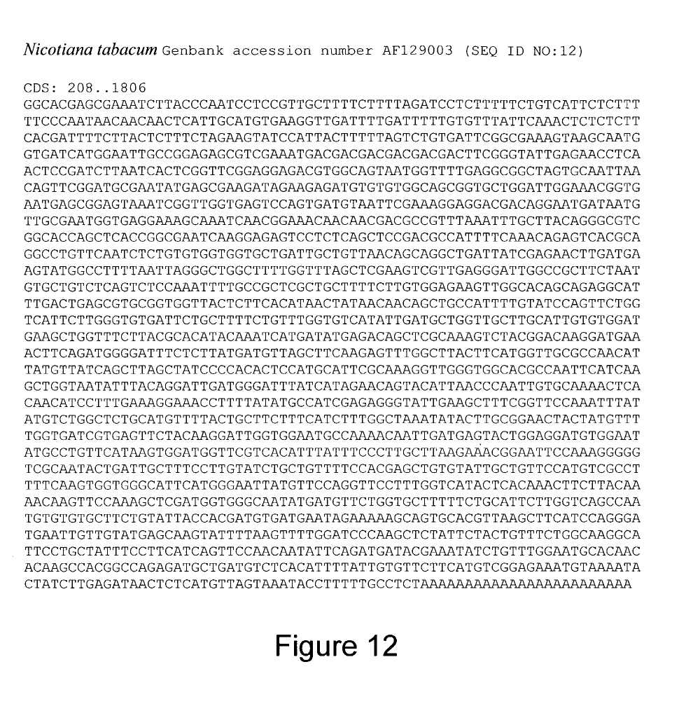

FIG. 13 shows Nicotiana tabacum Genbank accession number AAF19345 (SEQ ID NO:13)

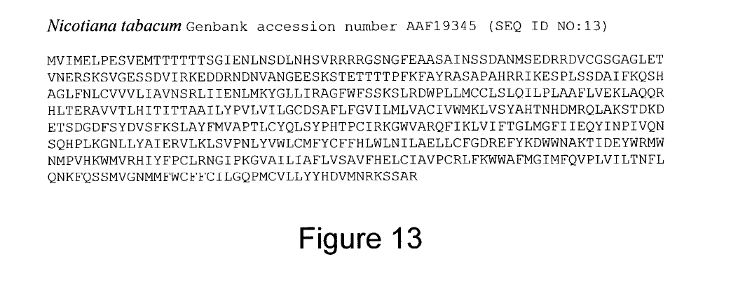

FIG. 14 shows Perilla frutescens Genbank accession number AF298815 (SEQ ID NO:14)

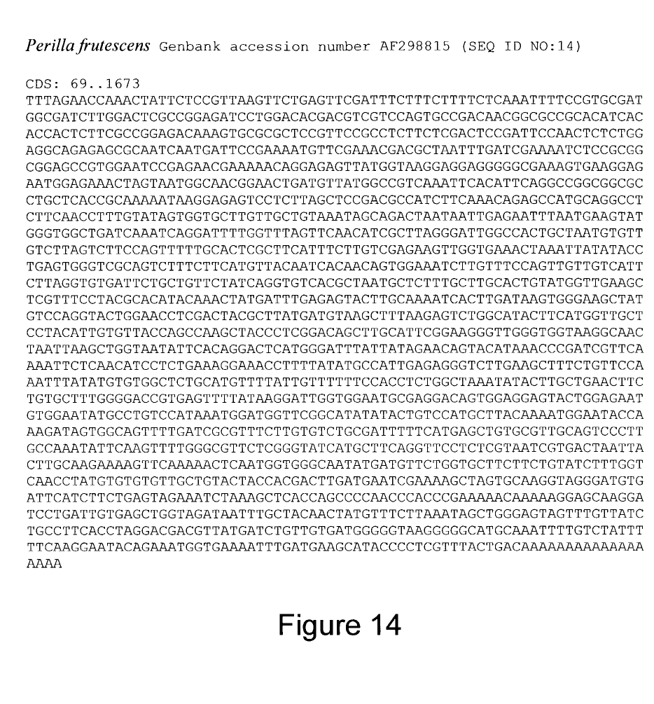

FIG. 15 shows Perilla frutescens Genbank accession number AAG23696 (SEQ ID NO:15)

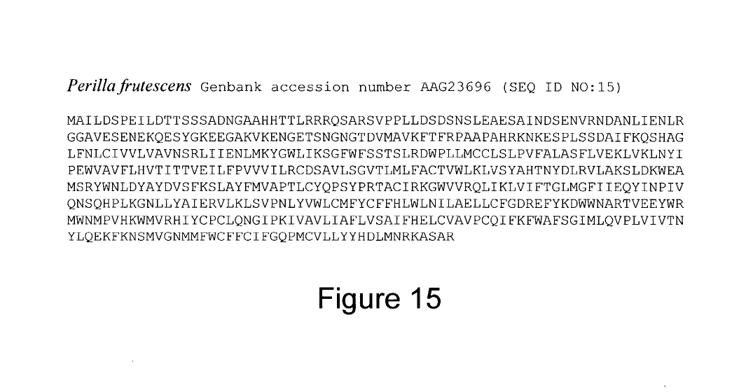

FIG. 16 shows Zea mays Genbank accession number AY110660 (SEQ ID NO:16)

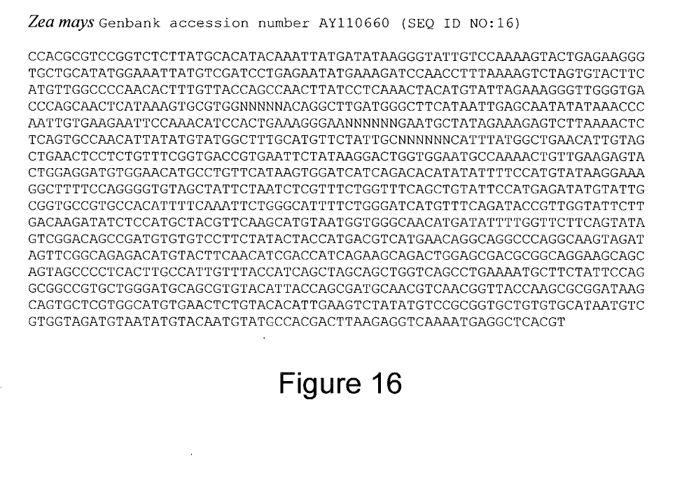

FIG. 17 shows Zea mays Genbank accession number PCO148220 (SEQ ID NO: 17)

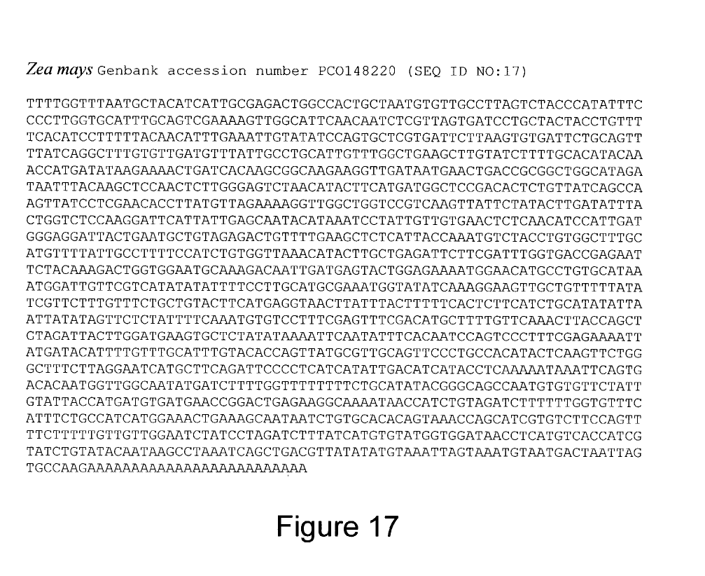

### DETAILED DESCRIPTION OF THE INVENTION

Plant nucleic acid compositions encoding polypeptide products with diacylglyceride acyltransferase (DGAT) activity, as well as the polypeptide products encoded thereby and methods for producing the same, are provided. Methods and compositions for modulating DGAT activity in a plant, particularly DGAT activity in plant seeds, and transgenic plants with altered DGAT activity are provided. Such plants and seeds are useful in the production of human food and animal feedstuff and have several other industrial applications. Also provided are methods for making triglycerides and triglyceride compositions, as well as the compositions produced by these methods. The subject methods and compositions find use in a variety of different applications, including research, medicine, agriculture and industry.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

Plant DGAT Nucleic Acid Compositions

Plant nucleic acid compositions encoding polypeptide products, as well as fragments thereof, having diglyceride acetyltransferase (DGAT) activity are provided. By plant nucleic acid composition is meant a composition comprising a sequence of DNA having an open reading frame that encodes a plant DGAT polypeptide, i.e. a gene from a plant encoding a polypeptide having DGAT activity, and is capable, under appropriate conditions, of being expressed as a DGAT polypeptide. Also encompassed in this term are plant nucleic acids that are homologous or substantially similar or identical to the nucleic acids encoding DGAT polypeptides or proteins. Thus, the subject invention provides genes encoding plant DGAT, such as genes encoding monocot DGAT and homologs thereof and dicot DGAT and homologs thereof, as well as Arabidopsis, canola and corn DGATs and homologs thereof. In other words, both monocot and dicot genes encoding DGAT proteins are provided by the subject invention.

The source of plant DGAT-encoding nucleic acids may be any plant species, including both monocot and dicots, and in particular cells from agriculturally important plant species, including, but not limited to: crops such as soybean, wheat, corn, potato, cotton, rice, oilseed rape (including canola), sunflower, alfalfa, sugarcane, castor and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, spinach, squash, sweet corn, tobacco, tomato, watermelon, rosaceous fruits (such as apple, peach, pear, cherry and plum) and vegetable brassicas (such as broccoli, cabbage, cauliflower, brussel sprouts and kohlrabi). Other crops, fruits and vegetables whose cells may be targetted include barley, rye, millet, sorghum, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yarn, and sweet potato, Arabidopsis, beans, mint and other labiates. Woody species, such as pine, poplar, yew, rubber, palm, eucalyptus etc. and lower plants such as mosses, ferns, and algae are also sources. A source of DGAT genes may also be selected from various plant families including Brassicaceae, Compositae, Euphorbiaceae, Leguminosae, Linaceae, Malvaceae, Umbilliferae and Graminae. Of particular interest are DGAT genes from oilseed crops, such as canola, soybean and corn, and DGAT genes from jajoba, coconut and meadowfoam, Limnanthes alba.

In certain embodiments, the coding sequence of the Arabidopsis thaliana DGAT gene is of interest, i.e. the A. thaliana cDNA encoding the A. thaliana DGAT enzyme, includes or comprises a nucleic acid sequence substantially the same as or identical to that identified as SEQ ID NO:1 infra. In certain other embodiments, the nucleic acid sequence encoding the full length DGAT enzyme from A. thaliana (SEQ ID NO:2), and the nucleic acid sequences encoding DGAT enzymes from Brassica napus (canola; SEQ ID NOS :4, 6 and 8), Tropaeolum majus (nasturtium; SEQ ID NO:10), Perilla frutescens (perilla; SEQ ID NO:12), Nicotiana tabacum (tabacco; SEQ ID NO:14) and Zea mays (corn; SEQ ID NO:16 and SEQ ID NO:17) are of interest.

Between plant species, e.g., Arabidopsis and tomato, or corn and rice, homologs have substantial sequence similarity, e.g. at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10. Unless specified otherwise, all sequence identity values provided herein are determined using GCG (Genetics Computer Group, Wisconsin Package, Standard Settings, gap creation penalty 3.0, gap extension penalty 0.1). The sequences provided herein are essential for recognizing DGAT-related and homologous plant polynucleotides in database searches. A P-value cut-off, as determined by the output of a BLAST search, may also be used to determine whether a sequence in a database is a homolog. When a P-value cut-off is utilized, usually a homologs are identified with a P-value cut-off of 10⁻¹⁰ and below, 10⁻²⁰ and below, 10⁻³⁰ and below or 10⁻⁵⁰ and below.

Nucleic acids encoding the plant DGAT proteins and DGAT polypeptides of the subject invention may be cDNAs or genomic DNAs, as well as fragments thereof. The term “DGAT-gene” shall be intended to mean the open reading frame encoding specific plant DGAT proteins and polypeptides, and DGAT introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into a host genome.

The term “cDNA” as used herein is intended to include all plant nucleic acids that share the arrangement of sequence elements found in native mature mRNA species, where sequence elements are exons and 5′ and 3′ non-coding regions. Normally mRNA species have contiguous exons, with the intervening introns, when present, being removed by nuclear RNA splicing, to create a continuous open reading frame encoding a DGAT protein.

A genomic sequence of interest comprises the nucleic acid present between the initiation codon and the stop codon, as defined in the listed sequences, including all of the introns that are normally present in a native chromosome. It may further include the 5′ and 3′ untranslated regions found in the mature mRNA. It may further include specific transcriptional and translational regulatory sequences, such as promoters, enhancers, etc., including about 1 kb, but possibly more, of flanking genomic DNA at either the 5′ and 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acid compositions of the subject invention may encode all or a part of the subject plant DGAT proteins and polypeptides, described in greater detail infra. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt.

The plant DGAT-genes of the subject invention are isolated and obtained in substantial purity, generally as other than an intact chromosome. Usually, the DNA will be obtained substantially free of other nucleic acid sequences that do not include a DGAT sequence or fragment thereof, generally being at least about 50%, usually at least about 90% pure and are typically “recombinant”, i.e. flanked by one or more nucleotides with which it is not normally associated on a naturally occurring chromosome.

Also provided are nucleic acids that hybridize to the above described nucleic acids under stringent conditions, i.e. high or low stringency conditions. An example of high stringency hybridization conditions is hybridization overnight at about 50° C. in 0.1×SSC (15 mM sodium chloride/1.5 mM sodium citrate) followed by two 30 minute washes in 0.1×SSC at about 50° C. An example of low stringency conditions is hybridization overnight at about 50° C. in 2×SSC followed by two 30 minute washes in 2×SSC at about 50° C. Other stringent hybridization conditions are known in the art and may also be employed to identify nucleic acids of this particular embodiment of the invention. For example, other high stringency hybridization conditions include overnight incubation at temperatures other than 50° C., or overnight incubation at 42° C. in a solution containing 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Usually the nucleic acids that hybridize to the above described nucleic acids are derived from plants, particularly libraries of plant polynucleotides from monocots or dicots, and particularly libraries of the species listed above.

Also provided are nucleic acids that encode the proteins encoded by the above described nucleic acids, but differ in sequence from the above described nucleic acids due to the degeneracy of the genetic code.

In addition to the plurality of uses described in greater detail in following sections, the subject nucleic acid compositions find use in the preparation of all or a portion of the plant DGAT polypeptides, as described below.

Plant DGAT Polypeptide Compositions

Also provided by the subject invention are plant polypeptides having DGAT activity, i.e. capable of catalyzing the acylation of diacylglycerol. In certain embodiments and in addition to being capable of catalyzing the esterification of diacylglycerol with a fatty acyl CoA substrates, the subject proteins are incapable of esterifying, at least to any substantial extent, the following substrates: cholesterol, 25-hydroxy-, 27-hydroxy-,7α-hydroxy- or 7β-hydroxycholesterols, 7-ketocholesterol, vitamins D2 and D3, vitamin E, dehydrepiandrosterone, retinol, ethanol, sitosterol, lanosterol and ergosterol.

The term “polypeptide” composition as used herein refers to both the full length proteins as well as portions or fragments thereof. Also included in this term are variations of the naturally occurring proteins, where such variations are homologous or substantially similar to the naturally occurring protein, as described in greater detail below, be the naturally occurring protein the Arabidopsis protein, canola protein, or corn protein of from some other species which naturally expresses a DGAT enzyme, be that species monoct or dicot. In the following description of the subject invention, the term DGAT is used to refer not only to the Arabidopsis form of the enzyme, but also to homologs thereof expressed in non-Arabidopsis species, including other dicot species such as canola and monocot species such as corn.

The subject plant DGAT proteins are, in their natural environment, trans-membrane proteins. The subject proteins are characterized by the presence of at least one potential N-linked glycosylation site, at least one potential tyrosine phosphorylation site, and multiple hydrophobic domains, including 6 to 12 hydrophobic domains capable of serving as trans-membrane regions. The proteins range in length from about 300 to 650, usually from about 450 to 550 and more usually from about 500 to 550 amino acid residues, and the projected molecular weight of the subject proteins based solely on the number of amino acid residues in the protein ranges from about 40 to 80, usually from about 45 to 75 and more usually from about 50 to 65 kDa, where the actual molecular weight may vary depending on the amount of glycolsylation of the protein and the apparent molecular weight may be considerably less (e.g. 40 to 50 kDa) because of SDS binding on gels.

The amino acid sequences of the subject proteins are characterized by having at least some homology to a corresponding ACAT protein from the same species, e.g. an Arabidopsis DGAT protein has at least some sequence homology with the ACAT protein, the corn DGAT protein has at least some sequence homology with the mouse ACAT-1 corn, etc., where the sequence homology will not exceed about 50%, and usually will not exceed about 40% and more usually will not exceed about 25%, but will be at least about 15% and more usually at least about 20%, as determined using GCG (Genetics Computer Group, Wisconsin Package, Standard Settings, Gap Creation Penalty 3.0, Gap Extension Penalty 0.1).

Of particular interest in many embodiments are plant DGAT proteins that are non-naturally glycosylated. By non-naturally glycosylated is meant that the protein has a glycosylation pattern, if present, which is not the same as the glycosylation pattern found in the corresponding naturally occurring protein. For example, human DGAT of the subject invention and of this particular embodiment is characterized by having a glycosylation pattern, if it is glycosylated at all, that differs from that of naturally occurring human DGAT. Thus, the non-naturally glycosylated DGAT proteins of this embodiment include non-glycosylated DGAT proteins, i.e. proteins having no covalently bound glycosyl groups.

In addition to the specific DGAT proteins described above, homologs or proteins (or fragments thereof) from other species, i.e. monocot and dicot plant species, are also provided, where such homologs or proteins may be from a variety of different types of species, including both monocot and dicots and in particular from agriculturally important plant species, including, but not limited to, crops such as soybean, wheat, corn, potato, cotton, rice, oilseed rape (including canola), sunflower, alfalfa, sugarcane, castor and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, spinach, squash, sweet corn, tobacco, tomato, watermelon, rosaceous fruits (such as apple, peach, pear, cherry and plum) and vegetable brassicas (such as broccoli, cabbage, cauliflower, brussel sprouts and kohlrabi). Other crops, fruits and vegetables whose cells may be targetted include barley, rye, millet, sorghum, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yarn, and sweet potato, Arabidopsis, beans, mint and other labiates. Woody species, such as pine, poplar, yew, rubber, palm, eucalyptus etc. and lower plants such as mosses, ferns, and algae are also sources. A source of homologous plant genes may also be selected from various plant families including Brassicaceae, Compositae, Euphorbiaceae, Leguminosae, Linaceae, Malvaceae, Umbilliferae and Graminae. Of particular interest are homologous genes from oilseed crops, such as canola, soybean and corn. By homolog is meant a protein having at least about 35%, usually at least about 40% and more usually at least about 60% amino acid sequence identity the specific DGAT proteins as identified in SEQ ID NOS: 04 to 06, where sequence identity is determined using GCG, supra.

Of particular interest in certain embodiments is the Arabidopsis DGAT protein, where the Arabidopsis DGAT protein of the subject invention has an amino acid sequence that comprises or includes a region substantially the same as or identical to the sequence appearing as SEQ ID NO:3 infra. As such, DGAT proteins having an amino acid sequence that is substantially the same as or identical to the sequence of SEQ ID NO:3 are of interest. By substantially the same as is meant a protein having a region with a sequence that has at least about 75%, usually at least about 90% and more usually at least about 98% sequence identity with the sequence of SED ID NO:3, as measured by GCG, supra. Of particular interest in other embodiments are the DGAT proteins from canola (SEQ ID NOS:5-9), nasurtium (SEQ ID NO:11), perilla (SEQ ID NO:13), tobacco (SEQ ID NO:15) and corn (encoded by nucleotides comprising SEQ ID NO:16 or SEQ ID NO:17). Also of particular interest in yet other embodiments of the subject invention is the A. thaliana DGAT protein (SEQ ID NO:3), where the A. thaliana DGAT protein of the subject invention has an amino acid sequence encoded by the nucleic acid comprising the sequence appearing as SEQ ID NO:1, infra.

The plant DGAT proteins of the subject invention (e.g. Arabidopsis DGAT or a homolog thereof, non-Arabidopsis DGAT proteins, e.g. canola DGAT, corn DGAT etc) are present in a non-naturally occurring environment, e.g. are separated from their naturally occurring environment. In certain embodiments, the subject DGAT is present in a composition that is enriched for DGAT as compared to DGAT in its naturally occurring environment. As such, purified DGAT is provided, where by purified is meant that DGAT is present in a composition that is substantially free of non DGAT proteins, where by substantially free is meant that less than 90%, usually less than 60% and more usually less than 50% of the composition is made up of non-DGAT proteins. For compositions that are enriched for DGAT proteins, such compositions will exhibit a DGAT activity of at least about 100, usually at least about 200 and more usually at least about 1000 pmol triglycerides formed/mg protein/min, where such activity is determined by the assay described in the Experimental Section, of U.S. patent application Ser. No. 10/040,315.

In certain embodiments of interest, the plant DGAT protein is present in a composition that is substantially free of the constituents that are present in its naturally occurring environment. For example, a plant DGAT protein comprising composition according to the subject invention in this embodiment will be substantially, if not completely, free of those other biological constituents, such as proteins, carbohydrates, lipids, etc., with which it is present in its natural environment. As such, protein compositions of these embodiments will necessarily differ from those that are prepared by purifying the protein from a naturally occurring source, where at least trace amounts of the proteins constituents will still be present in the composition prepared from the naturally occurring source.

The plant DGAT of the subject invention may also be present as an isolate, by which is meant that the DGAT is substantially free of both non-DGAT proteins and other naturally occurring biologic molecules, such as oligosaccharides, polynucleotides and fragments thereof, and the like, where substantially free in this instance means that less than 70%, usually less than 60% and more usually less than 50% of the composition containing the isolated DGAT is a non-DGAT naturally occurring biological molecule. In certain embodiments, the DGAT is present in substantially pure form, where by substantially pure form is meant at least 95%, usually at least 97% and more usually at least 99% pure.

In addition to the naturally occurring plant DGAT proteins, DGAT polypeptides which vary from the naturally occurring DGAT proteins are also provided. By DGAT polypeptides is meant proteins having an amino acid sequence encoded by an open reading frame (ORF) of a plant DGAT gene, described supra, including the full length DGAT protein and fragments thereof, particularly biologically active fragments and/or fragments corresponding to functional domains; and including fusions of the subject polypeptides to other proteins or parts thereof. Fragments of interest will typically be at least about 10 aa in length, usually at least about 50 aa in length, and may be as long as 300 aa in length or longer, but will usually not exceed about 1000 aa in length, where the fragment will have a stretch of amino acids that is identical to a DGAT protein of SEQ ID NOS: 3, 5, 7, 9, 11 or 13 or a homolog thereof, of at least about 10 aa, and usually at least about 15 aa, and in many embodiments at least about 50 aa in length.

Preparation of Plant DGAT Polypeptides

The subject plant DGAT proteins and polypeptides may be obtained from naturally occurring sources, but are preferably synthetically produced. Where obtained from naturally occurring sources, the source chosen will generally depend on the species from which the DGAT is to be derived.

The subject DGAT polypeptide compositions may be synthetically derived by expressing a recombinant gene encoding DGAT, such as the polynucleotide compositions described above, in a suitable host. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a DGAT gene, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the gene. After introduction of the DNA, the cells containing the construct may be selected by means of a selectable marker, the cells expanded and then used for expression.

DGAT proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, the oeogenic filamentous fungus Mortierella ramanniana, insect cells in combination with baculovirus vectors, or cells of a higher organism such as plants, particularly monocots and dicots, e.g. Z. mays or tobacco cells, may be used as the expression host cells. In some situations, it is desirable to express the DGAT gene in eukaryotic cells, where the DGAT protein will benefit from native folding and post-translational modifications. Small peptides can also be synthesized in the laboratory. Polypeptides that are subsets of the complete DGAT sequence may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol (1986) 6:142; Kunze et al., J. Basic Microbiol. (1985) 25:141; Gleeson et al., J. Gen. Microbiol. (1986) 132-3459; Roggenkamp et al., Mol. Gen. Genet. (1986) 202:302; Das et al., J. Bacteriol. (1984) 158:1165; De Louvencourt et al., J. Bacteriol. (1983) 154.737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. Pat. No. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism.

Once the source of the protein is identified and/or prepared, e.g. a transfected host expressing the protein is prepared, the protein is then purified to produce the desired DGAT comprising composition. Any convenient protein purification procedures may be employed, where suitable protein purification methodologies are described in Guide to Protein Purification, (Deuthser ed.) (Academic Press, 1990). For example, a lysate may prepared from the original source, e.g. naturally occurring cells or tissues that express DGAT or the expression host expressing DGAT, and purified using HPLC, exclusion chromatography, gel electrophoresis, affinity chromatography, and the like.

Once the gene corresponding to a selected polynucleotide is identified, its expression can be regulated in the cell to which the gene is native. For example, an endogenous gene of a cell can be regulated by an exogenous regulatory sequence introduced into the cell by site specific insertion, e.g., by homologous recombination. Further methods for modulating the expression of an endogenous gene include creating a library of plants each containing an endogenous modulating element (for example an activating T-DNA or transposable element) inserted at a different position in the plant's genome and screening the library for plants with modulated expression of the endogenous gene. A number of methods for modulating the expression of an endogenous gene of a cell are disclosed in U.S. Pat. Nos. 5,641,670, 5,968,502, 5,994,127, and 6,355,241, and in Plant Cell. (2000) 12: 2383-2394; Development (2000) 127: 4971-80; Plant Cell (2000) 12:1619-32; Science. (1999) 286:1962-5; Plant J. (1885) 359-365; Kempin et al. (1997) Nature 889:802-808; Plant Mol. Biol (2002) 48: 183-200, the disclosures of which is herein incorporated by reference.

Antibodies

Also of interest are antibodies that detect plant DGAT protein. Suitable antibodies are obtained by immunizing a host animal with peptides comprising all or a portion of a plant DGAT protein, such as the DGAT polypeptide compositions of the subject invention. Suitable host animals include mouse, rat sheep, goat, hamster, rabbit, etc. The origin of the protein immunogen may be monocot or dicot DGAT, from Arabidopsis, canola, nasturtium, tabacco or corn etc.

The immunogen may comprise the complete protein, or fragments and derivatives thereof. Preferred immunogens comprise all or a part of DGAT, where these residues contain the post-translation modifications, such as glycosylation, found on the native DGAT. Immunogens comprising the extracellular domain are produced in a variety of ways known in the art, e.g. expression of cloned genes using conventional recombinant methods, isolation from HEC, etc.

For preparation of polyclonal antibodies, the first step is immunization of the host animal with plant DGAT, where the DGAT will preferably be in substantially pure form, comprising less than about 1% contaminant. The immunogen may comprise complete DGAT, fragments or derivatives thereof. To increase the immune response of the host animal, the DGAT may be combined with an adjuvant, where suitable adjuvants include alum, dextran, sulfate, large polymeric anions, oil & water emulsions, e.g. Freund's adjuvant, Freund's complete adjuvant, and the like. The DGAT may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The DGAT is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using DGAT bound to an insoluble support, protein A sepharose, etc.

The antibody may be produced as a single chain, instead of the normal multimeric structure. Single chain antibodies are described in Jost et al. (1994) J.B.C. 269:26267B73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CHl domain and hinge region of the H chain, followed by a translational stop codon to yield the truncated molecule.

Modifying DGAT Expression and/or Activity in a Plant

Also provided are methods of modifying DGAT expression and/or activity in a plant. In several embodiments, plant DGAT-encoding polynucleotides are used to modify DGAT activity in a plant to produce a variety of trait-modified plants, including plants with altered TAG levels, altered TAG compositions, or altered total seed protein content, particularly in seeds.

Several strategies may be employed to modify DGAT activity in a plant, including those that increase DGAT activity, and those that decrease DGAT activity.

Expression of a DGAT Transgene in a Plant

Typically, plant DGAT polynucleotide sequences of the invention are incorporated into recombinant nucleic acid, e.g. DNA or RNA, molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homologue.

The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

In general, expression plasmids will contain a selectable marker and DGAT nucleic acid sequences. The selectable marker provides resistance to toxic chemicals and allows selection of cells containing the marker over cells not containing the marker. Conveniently, the marker encodes resistance to a herbicide, e.g. phosphinothricin, glyphosate etc, or an antibiotic, such as kanamycin, G418, bleomycin, hygromycin, chloramphenicol, or the like. The particular marker employed is one that allows for selection of transformed cells over cells lacking the introduced recombinant DNA. Antibiotic or herbicide resistance markers including cat (chloramphenicol acetyl transferase), npt II (neomycin phosphotransferase II), PAT (phosphinothricin acetyltransferase), ALS (acetolactate synthetase), EPSPS (5-enolpyruvyl-shikimate-3-phosphate synthase), and bxn (bromoxynil-specific nitrilase) may be used. A preferred marker sequence is a DNA sequence encoding a selective marker for herbicide resistance and most particularly a protein having enzymatic activity capable of inactivating or neutralizing herbicidal inhibitors of glutamine synthetase. The non-selective herbicide known as glufosinate (BASTA™ or LIBERTY™) is an inhibitor of the enzyme glutamine synthetase. It has been found that naturally occurring genes or synthetic genes can encode the enzyme phosphinothricin acetyl transferase (PAT) responsible for the inactivation of the herbicide. Such genes have been isolated from Streptomyces. Specific species include Streptomyces hygroscopicus (Thompson C. J. et al., EMBO J., vol. 6:2519-2523 (1987)), Streptomyces coelicolor (Bedford et al, Gene 104: 39-45 (1991)) and Streptomyces viridochromogenes (Wohlleben et al. Gene 80:25-57 (1988)). These genes including those that have been isolated or synthesized are also frequently referred to as bar genes. These genes have been cloned and modified for transformation and expression in plants (EPA 469 273 and U.S. Pat. No. 5,561,236). Through the incorporation of the pat gene, corn plants and their offspring can become resistant against phosphinothricin (or glufosinate).

General texts that describe molecular biological techniques useful herein, including the use and production of vectors, promoters and many other relevant topics, include Berger, Sambrook and Ausubel, supra. Any of the identified sequences can be incorporated into a cassette or vector, e.g., for expression in plants. A number of expression vectors suitable for stable transformation of plant cells or for the establishment of transgenic plants have been described including those described in Weissbach and Weissbach, (1989) Methods for Plant Molecular Biology, Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303:209, Bevan (1984) Nucl Acid Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou(1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol 102: 1077-1084; Vasil (1993) Bio/Technology 10: 667-674; Wan and Lemeaux (1994) Plant Physiol 104: 37-48, and for Agrobacterium-mediated DNA transfer (Ishida et al. (1996) Nature Biotech 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

Examples of constitutive plant promoters which can be useful for expressing a DGAT sequence include: the cauliflower mosaic virus (CaMV) 35S promoter, which confers constitutive, high-level expression in most plant tissues (see, e.g., Odel et al. (1985) Nature 313:810); the nopaline synthase promoter (An et al. (1988) Plant Physiol 88:547); and the octopine synthase promoter (Fromm et al. (1989) Plant cell 1:977).

A variety of plant gene promoters that regulate gene expression in response to environmental, hormonal, chemical, developmental signals, and in a tissue-active manner can be used for expression of DGAT sequence in plants. Choice of a promoter is based largely on the phenotype of interest and is determined by such factors as tissue (e.g., seed, fruit, root, pollen, vascular tissue, flower, carpet, etc.), inducibility (e.g., in response to wounding, heat, cold, drought, light, pathogens, etc.), timing, developmental state, and the like. Numerous known promoters have been characterized and can favorable be employed to promote expression of a polynucleotide of the invention in a transgenic plant or cell of interest. For example, fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol Biol 11:651), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998) Plant Mol Biol 37:977-988), flower-specific (Kaiser et al, (1995) Plant Mol Biol 28:231-243), pollen (Baerson et al. (1994) Plant Mol Biol 26:1947-1959), carpels (Ohl et al. (1990) Plant Cell 2:837-848), pollen and ovules (Baerson et al. (1993) Plant Mol Biol 22L255-267), auxin-inducible promoters (such as that described in van der Kop et al. (1999) Plant Mol Biol 39:979-990 or Baumann et al. (1999) Plant Cell 11:323-334), cytokinin-inducible promoter (Guevara-Garcia (1998) Plant Mol Biol 38: 743-753), promoters responsive to gibberellin (Shi et al. (1998) Plant Mol Biol 38: 1053-1060, Willmott et al. (1998) 38:817-825) and the like. Additional promoters are those that elicit expression in response to heat (Ainley et al. (1993) Plant Mol Biol 22: 13-23), light (e.g., the pea rbcS-3A promoter, Kuhlemeier et al. (1989) Plant Cell 1:471, and the maize rbcS promoter, Schaffner and Sheen (1991) Plant Cell 3: 997); wounding (e.g., wunI, Siebertz et al. (1989) Plant Cell 1:961); pathogens (such as the PR-1 promoter described in Buchel et al. (1999) Plant Mol. Biol. 40:387-396, and the PDF1.2 promoter described in Manners et al. (1998) Plant Mol. Biol. 38: 1071-80), and chemicals such as methyl jasmonate or salicylic acid (Gatz et al. (1997) Plant Mol Biol 48: 89-108). In addition, the timing of the expression can be controlled by using promoters such as those acting at senescence (An and Amaxon (1995) Science 270: 1986-1988); or late seed development (Odell et al. (1994) Plant Physiol 106:447-458). For modification of seed traits, seed specific expression of DGAT is preferable. This can be accomplished using one of the many seed-specific promoters available to one of skill in the art, including the seed-specific promoters of the napin, phaseolin, cruciferin, oleosin, oleate 12-hydroxylase (Plant J. (1998) 13:201-10), DC3 (U.S. Pat. No. 5,773,697) genes, etc.

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Specific initiation signals can aid in efficient translation of coding sequences. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and inititation codons can be of various origins, both natural and synthetic The efficiency of expression can be enhanced by the inclusion of enhancers appropriate to the cell system in use.

Reduction of Endogenous Gene Expression

It is often desirable to reduce the expression of DGAT in a plant to reduce the amount of TAG or increase the amount of total protein, particularly in seeds. The subject nucleic acids may be used in many methods to reduce DGAT activity, several of which methods are described below. Exemplary methods for reducing activity of DGAT plant are sub-divided into gene “silencing” and “knock-out” strategies.

Methods for gene silencing, including antisense, RNAi, ribozyme and cosuppression technologies are based in hybridization of an expressed exogenous nucleic acid with an RNA transcribed from an endogenous gene of interest in a plant cell. Because the methods are based on hybridization, the methods are particularly applicable to silencing of gene families that share a level of sequence identity, for example for families of genes that contain 60% or more, 70% or more, 80% or more, 90% or more or 95% or more sequence identity over 100, 200, or 500 or more nucleotides. RNA-induced silencing strategies for plants are reviewed in Matzke et al (Curr Opin Genet Dev. 2001 11:221-7). Such cells may be used to decrease the endogenous levels of DGAT in a plant.

Antisense, Cosuppression and RNAi Approaches

In addition to expression of the plant DGAT nucleic acids of the invention as plant phenotype modification nucleic acids, the nucleic acids are also useful for sense and anti-sense suppression of expression, e.g., to down-regulate expression of DGAT, e.g., as a further mechanism for modulating plant phenotype. That is, the nucleic acids of the invention, or subsequences or anti-sense sequences thereof, can be used to block expression of naturally occurring DGAT nucleic acids. A variety of sense and anti-sense technologies are known in the art, e.g., as set forth in Lichtenstein and Nellen (1997) Antisense Technology: A Practical Approach IRL Press at Oxford University, Oxford, England. In general, sense or antisense sequences are introduced into a cell, where they are optionally amplified, e.g., by transcription. Such sequences include both simple oligonucleotide sequences and catalytic sequences such as ribozymes.

For example, a reduction or elimination of expression (i.e., a “knock-out”) of a DGAT homologous gene in a transgenic plant, e.g., to decrease TAG levels, can be obtained by introducing an antisense construct containing a DGAT cDNA. For antisense suppression, the DGAT cDNA is arranged in reverse orientation (with respect to the coding sequence) relative to the promoter sequence in the expression vector. The introduced sequence need not be the full length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater that 100 nucleotides. Transcription of an antisense construct as described results in the production of RNA molecules that are the reverse complement of mRNA molecules transcribed from the endogenous transcription factor gene in the plant cell.

Suppression of endogenous DGAT gene expression can also be achieved using a ribozyme. Ribozymes are RNA molecules that possess highly specific endoribonuclease activity. The production and use of ribozymes are disclosed in U.S. Pat. No. 4,987,071 and U.S. Pat. No. 5,543,508. Synthetic ribozyme sequences including antisense RNAs can be used to confer RNA cleaving activity on the antisense RNA, such that endogenous mRNA molecules that hybridize to the antisense RNA are cleaved, which I turn leads to an enhanced antisense inhibition of endogenous gene expression.

Vectors in which RNA encoded by a DGAT cDNA is over-expressed can also be used to obtain co-suppression of a corresponding endogenous gene, e.g., in the manner described in U.S. Pat. No. 5,231,020 to Jorgensen. Such co-suppression (also termed sense suppression) does not require that the entire DGAT cDNA be introduced into the plant cells, nor does it require that the introduced sequence be exactly identical to the endogenous DGAT. However, as with antisense suppression, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of the DGAT mRNA, e.g., sequences comprising one or more stop codon, or nonsense mutation) can also be used to suppress expression of an endogenous DGAT, thereby reducing or eliminating it's activity and modifying one or more traits. Methods for producing such constructs are described in U.S. Pat. No. 5,583,021. Preferably, such constructs are made by introducing a premature stop codon into the DGAT gene.

Alternatively, DGAT gene expression can be modified by gene silencing using double-strand RNA (Sharp (1999) Genes and Development 13: 139-141). RNAi, otherwise known as double-stranded RNA interference (dsRNAi), has been extensively documented in the nematode C. elegans (Fire, A., et al, Nature, 391, 806-811, 1998) and an identical phenomenon occurs in plants, in which it is usually referred to as post-transcriptional gene silencing (PTGS) (Van Blokland, R., et al., Plant J., 6: 861-877, 1994; deCarvalho-Niebel, F., et at, Plant Cell, 7: 347-358, 1995; Jacobs, J. J. M. R. et al., Plant J., 12: 885-893, 1997; reviewed in Vaucheret, H., et al., Plant J., 16: 651-659, 1998). The phenomenon also occurs in fungi (Romano, N. and Masino, G., Mol. Microbiol., 6: 3343-3353, 1992, Cogoni, C., et al., EMBO J., 15: 3153-3163; Cogoni, C. and Masino, G., Nature, 399: 166-169, 1999), in which it is often referred to as “quelling”. RNAi silencing can be induced many ways in plants, where a nucleic acid encoding an RNA that forms a “hairpin” structure is employed in most embodiments. Alternative strategies include expressing RNA from each end of the encoding nucleic acid, making two RNA molecules that will hybridize. Current strategies for RNAi induced silencing in plants are reviewed by Carthew et al (Curr Opin Cell Biol. 2001 13:244-8).

Another method for abolishing the expression of a DGAT gene is by insertion mutagenesis using the T-DNA of Agrobacterium tumefaciens. After generating the insertion mutants, the mutants can be screened to identify those containing the insertion in a DGAT gene. Plants containing a single transgene insertion event at the desired gene can be crossed to generate homozygous plants for the mutation (Koncz et al. (1992) Methods in Arabidopsis Research, World Scientific).

Alternatively, a plant phenotype can be altered by eliminating an endogenous DGAT, e.g., by homologous recombination (Kempin et al. (1997) Nature 389:802). A DGAT gene can also be modified by using the cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The plant DGAT polynucleotides and polypeptides of this invention can also be expressed in a plant in the absence of an expression cassette by manipulating the activity or expression level of the endogenous gene by other means. For example, by ectopically expressing a gene by T-DNA activation tagging (Ichikawa et al. (1997) Nature 390 698-701; Kakimoto et al. (1996) Science 274:982-985). This method entails transforming a plant with a gene tag containing multiple transcriptional enhancers and once the tag has inserted into the genome, expression of a flanking gene coding sequence becomes deregulated. In another example, the transcriptional machinery in a plant can be modified so as to increase transcription levels of a polynucleotide of the invention (See, e.g., PCT Publication WO 96/06166 and WO 98/53057, which describe the modification of the DNA binding specificity of zinc finger proteins by changing particular amino acids in the DNA binding motif).

Plant Transformation

Transgenic plants (or plant cells, or plant explants, or plant tissues) incorporating the DGAT polynucleotides of the invention and/or expressing the DGAT polypeptides of the invention can be produced by a variety of well established techniques. Following construction of a vector, most typically an expression cassette, including a polynucleotide, e.g., encoding a DGAT or DGAT homolog of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture-Crop Species. Macmillan Publ. Co. Shimamoto et al. (1989) Nature 338:274-276, Fromm et al. (1990) Bio/Technology 8:833-839; and Vasil et al. (1990) Bio/Technology 8:429-434.

Transformation and regeneration of both monocotyledonous ad dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplast; liposome-mediated transformation; polyethylene glycol (PEG) mediated transformation; transformation using viruses: micro-injection of plant cells; micro-projectile bombardment of plant cells; vacuum infiltration; whiskers technology, and Agrobacterium tumeficiens mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified. The modified trait can be any of those traits described below. Additionally, to confirm that the modified trait is due to changes in expression levels or activity of the polypeptide or polynucleotide of the invention can be determined by analyzing mRNA expression using Northern blots, RT-PCR or microarrays, or protein expression using immunoblots or Western blots or gel shift assays.

Utility

The above described methods and compositions find use in a variety of different applications, particularly in agricultural and food industries. In most embodiments, plant DGAT expression and/or activity is modified in a transgenic plant causing a modification in TAG composition or content of the transgenic plant, or a tissue thereof. Transgenic plants are usually processed, and used as an animal feedstuff, or may be used for human consumption, e.g., in a vegetable oil. In addition, TAG may be purified from transgenic plants and may be used as is or modified to be a component of many industrial compositions, such as biofuel, industrial lubricant, surfactant, paint, varnish, wax, soap, cosmetic and wax compositions. Plant DGAT may also be expressed in a non-plant host and used as an “industrial enzyme”, catalyzing the synthesis of TAG in vitro. Several such utilities are described in further detail below.

Of interest for use in producing triglyceride compositions are transgenic plant that have been genetically manipulated using the nucleic acid compositions of the subject invention to produce triglycerides and/or compositions thereof in one or more desirable ways. Transgenic plants of the subject invention are those plants that at least: (a) produce more triglyceride or triglyceride composition than wild type, e.g. produce more oil, such as by producing seeds having a higher oil content, as compared to wild-type; (b) produce less triglyceride or triglyceride composition than wild type, e.g. produce less oil, such as by producing seeds having a lower oil content, as compared to wild-type; (c) produce triglyceride compositions, e.g. oils, that are enriched for triglycerides and/or enriched for one or more particular triglycerides as compared to wild type; (d) produce more total seed protein as compared to wild type; (e) produce less total seed protein or seed protein composition as compared to wild type; and the like.

Of interest are transgenic plants that produce commercially valuable triglyceride compositions or oils, such as canola, rapeseed, palm, corn, etc., containing various poly- and mono-unsaturated fatty acids, and the like. Of particular interest are transgenic plants, such as canola, rapeseed, palm, oil, etc., which have been genetically modified to produce seeds having higher oil content than the content found in the corresponding wild type, where the oil content of the seeds produced by such plants is at least 10% higher, usually at least 20% higher, and in many embodiments at least 30% higher than that found in the wild type, where in many embodiments seeds having oil contents that are 50% higher, or even greater, as compared to seeds produced by the corresponding wild-type plant, are produced. The seeds produced by such DGAT transgenic plants can be used as sources of oil or as sources of additional DGAT transgenic plants. Such transgenic plants and seeds therefore find use in methods of producing oils. In such methods, DGAT transgenic plants engineered to produce seeds having a higher oil content than the corresponding wild-type, e.g. seeds in which the DGAT gene is overexpressed, are grown, the seeds are harvested and then processed to recover the oil. The subject transgenic plants can also be used to produce novel oils characterized by the presence of triglycerides in different amounts and/or ratios than those observed in naturally occurring oils. The transgenic plants described above can be readily produced by those of skill in the art armed with the nucleic acid compositions of the subject invention. Of particular interest are transgenic plants that overexpress DGAT exogenous DGAT proteins that have a different substrate specificity to the endogenous DGAT, allowing the alteration triglyceride composition. In these embodiments, e.g. the nasturtium DGAT polynucleotide (SEQ ID NO:6) that has some specificity to longer chain acyl molecules may be used to make longer chain triglycerides.

In many embodiments, the trait modifications of particular interest include those to seed (such as embryo or endosperm), fruit, root, flower, pericarp, leaf, stem, shoot, seedling, entire plant or the like

The triglyceride compositions described above find use in a variety of different applications. For example, such compositions or oils find use as food stuffs, being used as ingredients, spreads, cooking materials, etc. Alternatively, such oils find use as industrial feedstocks for use in the production of chemicals, lubricants, surfactants, paints, varnishes, and biofuels and the like.

Modulators of Plant DGAT Expression and/or Activity

The plant DGAT polynucleotides and polypeptides of the present invention may also be used to identify endogenous or exogenous molecules that can modulate DGAT expression and/or activity in a plant to produce a DGAT trait modified plant. In one embodiment, such molecules include organic and inorganic molecules that interact with the DGAT enzyme and modulate its activity, and in other embodiments, the small molecule may modulate the expression of DGAT by, e.g., modulating the expression of DGAT-encoding mRNA.

Endogenous molecules that interact with and modulate plant DGAT enzyme may be identified by any method for detecting covalent modification, e.g., phosphorylation, or for detecting protein-protein interactions, including co-immunoprecipitation, cross-linking, co-purification through gradients or chromatographic columns, or by the yeast two hybrid system (Chien et al, 1991, Proc. Natl. Acad. Sci., 88: 9578-9582). Endogenous molecules that modulate plant DGAT expression may be identified by measuring DGAT gene expression, for example using mRNA levels as determined by a microarray or northern blot hybridization, by measuring the amount of DGAT polypeptide or its activity, or by measuring the activity of a DGAT promoter, in a library of plants that have been genetically altered, e.g., by insertional (by T-DNA, transposons, etc) or chemical (EMS, X-ray, etc) mutagenesis. Once a plant with altered expression and/or DGAT activity is identified, the endogenous factor may be genetically mapped or otherwise determined, isolated and/or cloned.

In addition to methods for identifying endogenous DGAT modulatory molecules described above, the DGAT polynucleotides and polypeptides also provide methods for identifying exogenous molecules that modulate activity or expression of DGAT in plants. In this embodiment, a test agent, usually a small or large molecule, is placed in contact with a plant cell (or a tissue, explant, entire plant, or other composition containing a plant cell) and a resulting effect on the cell is evaluated by monitoring, either directly or indirectly, one or more of the expression level of the DGAT polynucleotide (e.g. by RNA blot hybridization or microarrays) or polypeptide (e.g. by western blotting) or DGAT activity (by activity assay). In many cases, an alteration of a plant DGAT trait can be detected following contact of the plant with a modulatory molecule.

The following examples are offered primarily for purposes of illustration. It will be readily apparent to those skilled in the art that the formulations, dosages, methods of administration, and other parameters of this invention may be further modified or substituted in various ways without departing from the spirit and scope of the invention.

### EXPERIMENTAL

### Example I

Identification of DGAT cDNA from Arabidopsis thaliana.

The plant (Arabidopsis thaliana) DGAT gene (#AA042298) (SEQ ID NO:1) was identified from BLAST searches of the EST database using mouse DGAT sequences as a probe, as reported in U.S. patent application Ser. No. 09/103,754, the disclosure of which is herein incorporated by reference. The plant DGAT EST protein sequences encoded by plant DGAT genes are 40-50% identical to mammalianf DGAT enzymes. Furthermore, the plant DGAT sequences are more closely related to other mammalian DGAT sequences than to ACAT protein sequences.

### Example II

Identification of Other Plant DGAT Polynucleotides and Polypeptides.

Using the Arabidopsis DGAT nucleic acid sequence described by SEQ ID NO:1 as a probe of the GenBank nucleotide sequence database using the TBLASTX (V2.2.3) and TBLASTN (V2.2.3), further plant DGAT polynucleotides and polypeptides were identified: Arabidopsis DGAT polynucleotide #AJ238008 (SEQ ID NO:2) and encoded polypeptide (SEQ ID NO:3), Brassica napus polynucleotide #AF251794 (SEQ ID NO:4) and encoded polypeptide (SEQ ID NO:5), B. napus polynucleotide #AF155224 (SEQ ID NO:6) and encoded polypeptide (SEQ ID NO:7), B. napus polynucleotide # AF164434 (SEQ ID NO:8) and encoded polypeptide (SEQ ID NO:9), Tropaeolum majus polynucleotide # AY084052 (SEQ ID NO:10) and encoded polypeptide (SEQ ID NO:11), Nicotiana tabacum polynucleotide #AF129003 (SEQ ID NO:12) and encoded polypeptide (SEQ ID NO:13), Perilla frutescens polynucleotide #AF298815 (SEQ ID NO:14) and encoded polypeptide (SEQ ID NO 15), Zea mays polynucleotides #AY110660 and #PCO148220 (SEQ ID NOS 16 and 17, respectively).

### Example III

Increase of Oil Content in A. thaliana Seeds Expressing Plant DGAT Materials and Methods

Construction of DGAT cDNA Transformation Vector for Seed-Specific Expression: A full-length Arabidopsis thaliana DGAT cDNA (SEQ ID NO:2) is used as a template for PCR amplification with the primers DGATXbaI (CTAGTCTAGAATGGCGATTTTGGA) and DGATXhoI (GCGCTCGAGTTTCATGACATCGA) to provide new restriction sites on each end of the sequence. The PCR profile is as follows: 94° C. for 1 min; 30 cycles of 94° C. for 30 s, 55° C. for 30 s, 72° C. for 1 min; and 72° C. for 5 min. The PCR product is then ligated into the PCR-2.1 vector (Invitrogen, Carlsbad, Calif.). A 1.6-kb fragment is excised by a XbaI/KpnI digestion and ligated into the corresponding sites of the pSE. The plant transformation vector pSE is prepared from pRD400 (Datla et al., 1992 Gene 211: 383-384) by introducing a HindIII/XbaI fragment containing the B. napus napin promoter (Josefsson et al, 1987 J Biol Chem 262: 12196-12201) and a KpnI/EcoRI fragment containing the Agrobacterium nos terminator (Bevan, 1983 Acid Res 12: 8711-8721). The 1.6-kb DGAT cDNA fragment is ligated into XbaI/KpnI-digested pSE in the sense orientation. The resulting plasmid is designated napin:DGAT. Hence in the napin:DGAT construct, the Arabidopsis DGAT cDNA is under the control of the napin promoter. The construct integrity is confirmed by sequencing.

Transformation of Agrobacterium with Plant DGAT Vector Constructs: Electrocompetent Agrobacterium cells strain GV3101 (pMP90), are prepared and the Agrobacterium cells are transformed by electroporation with 20 to 50 ng of transforming DNA (napin:DGAT) according to the manufacturer's instructions, plated on a selective medium (Luria-Bertani broth with 50 μg mL1 kanamycin), and incubated for 48 h at 28° C. Single transformed cells are grown for 16 h (28° C., 225 rpm) in 5 mL Luria-Bertani broth with 50 μg mL1 kanamycin and 25 μg mL1 gentamycin. DNA extraction and purification are performed with a Qiaprep Spin Miniprep kit (Qiagen, Valencia, Calif.). The fidelity of the construct is rechecked by DNA sequencing before plant transformation.

Transformation of Arabidopsis: Seeds of Arabidopsis ecotype Columbia WT and mutant AS11 (Katavic et al., 1995) are grown at 22° C. under fluorescent illumination (120 μE m2 s1) in a 16-h-light/8-h-dark regime. Four to six plants typically are raised in a 10 cm2 pot in moistened Terra-lite Redi-earth (W. R. Grace and Company, Ajax, Ontario, Canada). To grow Agrobacterium, a 5-mL suspension in Luria-Bertani medium containing 50 μg mL1 kanamycin and 25 μg mL1 gentamycin is cultured overnight at 28° C. The day before infiltration, this “seed culture” is divided into four flasks containing 250 mL of Luria-Bertani medium supplemented with 50 μg mL1 kanamycin and 25 μg mL1 gentamycin. These culture are grown overnight at 28° C. Plants are vacuum infiltrated in an Agrobacterium suspension when the first flowers started opening.

The transformation is performed by vacuum infiltration using Silwet L-77 at a concentration of 0.005% in the dipping solution. The next day, the plants are uncovered, set upright, and allowed to grow for approximately 4 weeks in a growth chamber under continuous light conditions as described by Katavic et al. (1995). When the siliques are mature and dry, seeds are harvested and selected for positive transformants.

Selection of Putative Transformants (Transgenic Plants) and Analysis of Transgenic Plants and Seed Weights: For each construct, seeds are harvested in bulk. Seeds are surface-sterilized by submerging them in a solution containing 20% (v/v) bleach and 0.01% (v/v) Triton X-100 for 20 min, followed by three rinses with sterile water. Sterilized seeds are then plated by resuspending them in sterile 0.1% (w/v) phytagar at room temperature (approximately 1 mL phytagar for every 500-1,000 seeds), and then applying a volume containing 2,000 to 4,000 seeds onto 150×15-mm kanamycin selection plate. Plates are incubated for 2 d in the cold without light and then grown for 7 to 10 d in a controlled environment (22° C. under fluorescent illumination [120 μE m2 s1] in a 16-h-light/8-h-dark regime). The selection media contained one-half Murashige Skoog Gamborg medium, 0.8% (w/v) phytagar, 3% (w/v) Suc, 50 μg mL1 kanamycin, and 50 μg mL1 timentin. Petri dishes and lids are sealed with a Micropore surgical tape (3M Canada, Inc., London, ON, Canada). After 7 to 10 d, drug-resistant plants that had green leaves and well established roots within the medium are identified as T1 transformants, and at the 3- to 5-leaf stage selected transformants are transplanted into flats filled with heavily moistened soil mix. Transformants are grown to maturity, and mature seeds (T2 generation as defined in Katavic et al., 1994) are harvested from individual plants and further analyzed or propagated. Segregation analyses are performed on T2 plantlets screened on kanamycin to determine whether there are single (expected ratio of resistant:susceptible plantlets=3:1) or multiple copies of the napin:DGAT transgene. Homozygous single and multiple insert T2 lines exhibiting enhanced oil deposition and DGAT expression compared with one-dozen plasmid-only control transgenics are propagated to give T3 seed lines, for which further data on oil content, average seed weight, and yield per plant are collected. Average seed weights are determined from pooled T2 or individual T3 segregant seed lots and based upon six to eight individual samplings of 150 to 250 seeds/sample, with the seeds of each replicate being accurately counted on an Electronic Dual Light Transilluminator (Ultra Lum, Paramount, Calif.), using Scion Image software (Scion Corporation, Frederick, Md.). Weights and total oil content of the seeds of these samples are then individually recorded.

Results

The napin:DGAT plasmid is introduced into A. tumefaciens, used to transform wild-type Arabidopsis, and the progeny is analyzed. A number of primary napin:DGAT transgenic lines are produced, the T1 plantlets grown to maturity, and T2 seeds harvested. At the same time a number of independent plasmid only control transgenic (pSE vector without DGAT insert) lines, as well as non-transformed (n-t) WT and AS11 lines are propagated and analyzed.

On a mature seed dry weight basis, dry weight oil content of DGAT transgenic seeds is expected increase significantly.

### Example IV

Reduction of Oil Content in Brassica Plants Expressing Antisense DGAT

The Brassica napus cDNA described by SEQ ID NO:4 is used to design two PCR primers for amplification. The primers are: DGAT1 (CATCATCATCATACTGCCATGGACAGGTGTGATTCTGCTFTTTTATCA; SEQ ID NO:20) and DGAT2 (CTACTACTACTACTACTAGAGACAGGGCAATGTAGAAAGTATGTA; SEQ ID NO:21). A fragment of the DGAT gene is amplified from B. napus cv Westar genomic as follows: each PCR amplification is carried out in a 100 μl PCR reaction mixture containing 50 ng of B. napus genomic DNA, 200 μm of each dNTP, 1X buffer B (Gibco BRL), 1 μm of each primer, 3 mM magnesium sulfate and 2 μl Elongase enzyme (Gibco BRL). The reaction mixture is denatured at 94° C. for 3 minutes, followed by 30 cycles of denaturation at 94° C. for 1 minute, annealing at 50° C. for 2 minutes and extension at 72° C. for 3 minutes. A final extension incubation is performed at 72° C. for 10 minutes after cycling. A 1.4 kb fragment is amplified.

The amplified DNA fragment is cloned into pAMP1 vector (Gibco BRL), then excised using SmaI and SnaBI and ligated, in an antisense orientation, into pMB110, containing the seed specific cruciferin promoter and cruciferin termination sequences. This construct us used to transform Agrobacterium strain LBA4404, and the transformed Agrobacterium is used to transform B. napus cv. Westar. Regenerated plantlets are transferred to a greenhouse and grown to maturity. Each plant is self pollinated and seeds are harvested from each plant.

TAG is expected decrease significantly in the seeds of many of plants containing the DGAT antisense construct plants. Since a decrease in TAG levels is usually correlated with an increase in total cellular seed protein, a corresponding increase in total seed protein is also expected for these transgenic plants.

A similar experiment using an RNA interference strategy where the DGAT is expressed as a hairpin structure is predicted to give a more significant decrease in TAG levels.

It is evident from the above results and discussion that the subject invention provides an important new means for modulating TAG levels in plants Specifically, the subject invention provides a system for increasing or decreasing the levels of TAG in a plant seed. As such, the subject methods and systems find use in a variety of different applications, including research, industry, and the production of animal and human feedstuffs. Accordingly, the present invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A plant polynucleotide present in other than its naturally occurring environment encoding a product having DGAT activity.

2. The polynucleotide of claim 1, wherein said polynucleotide comprises a region of at least about 50 nucleotides in length that has at least about 75% sequence identity to a region of SEQ ID NO: 1.

3. The polynucleotide of claim 2, wherein said polynucleotide comprises a region of at least about 50 nucleotides in length that has at least about 90% sequence identity to a region of SEQ ID NO: 1.

4. The polynucleotide of claim 3, wherein said polynucleotide comprises a region of at least about 50 nucleotides in length that has at least about 95% sequence identity to a region of SEQ ID NO: 1.

5. The polynucleotide of claim 4, wherein said polynucleotide comprises a region of at least about 50 nucleotides in length that is identical to a region of SEQ ID NO: 1.

6. The isolated polynucleotide of claim 1, wherein said polynucleotide is a monocot polynucleotide

7. The isolated polynucleotide of claim 1, wherein said polynucleotide is a dicot polynucleotide

8. The isolated polynucleotide of claim 1, wherein said polynucleotide is a soybean, maize, or canola polynucleotide.

9. A nucleic acid that hybridizes under stringent conditions to the nucleic acid of SEQ ID NO: 1, wherein said nucleic acid encodes a DGAT polypeptide or is the complement of a nucleic acid that encodes a DGAT polypeptide.

10. A composition comprising a plant DGAT polypeptide present in other than its naturally occurring environment, wherein said composition exhibits a DGAT activity of at least about 100 pmol triglycerides formed/mg protein/min.

11. The DGAT polypeptide composition according to claim 10, wherein said polypeptide has an amino acid sequence of a naturally occurring DGAT polypeptide.

12. A fragment of a DGAT polypeptide.

13. The DGAT polypeptide fragment according to claim 12, wherein said polypeptide fragment is encoded by a polynucleotide comprising the sequence of SEQ ID NO:1.

14. An antibody binding specifically to a plant polypeptide having DGAT activity.

15. An expression cassette comprising the polynucleotide of claim 1.

16. A cell comprising an expression cassette of claim 15.

17. The cell according to claim 16, wherein said expression cassette is part of an extrachromosomal region or integrated into a chromosome of said cell.

18. The cell according to claim 16, wherein said cell is a plant cell.

19. A method of producing a polypeptide having plant DGAT activity, said method comprising:
a) growing a cell according to claim 16 under conditions sufficient to express said polypeptide; and
b) harvesting said polypeptide.

20. A transgenic plant comprising an expression cassette of claim 15.

21. The transgenic plant of claim 20, wherein at least one tissue of said plant is altered in DGAT activity as compared to wild type.

22. The transgenic plant according to claim 20, wherein said plant is modified in at least one DGAT trait.

23. The transgenic plant according to claim 22, wherein said DGAT trait is chosen from TAG content, TAG composition, DGAT protein content and total protein content.

24. The transgenic plant according to claim 22, wherein said trait is a seed trait.

25. The transgenic plant according to claim 24, wherein said seed trait is an increase in TAG content in seeds.

26. The transgenic plant according to claim 24, wherein said seed trait is an increase in total protein content in seeds.

27. Seeds of the transgenic plant according to claim 24.

28. A method to identify plant DGAT-encoding polynucleotides comprising:
employing a probe comprising a sequence substantially similar or identical to SEQ ID NO:1.

29. The method according to claim 28, wherein said probe is a nucleic acid molecule.

30. The method according to claim 29, wherein said probe is hybridized against a library of plant-derived nucleic acid fragments under stringent conditions.

31. The method according to claim 28, wherein said probe is a virtual probe.

32. The method according to claim 31, wherein said method comprises employing a nucleic acid sequence comparison program and a database comprising plant polynucleotide sequences.

33. The method according to claim 32, wherein said method employs a BLAST algorithm.

34. The method according to claim 33, wherein said BLAST algorithm is chosen from BLASTN and TBLASTX.

35. A method of producing a plant having a modified DGAT activity, the method comprising:
introducing an isolated polynucleotide of claim 1 into a cell of a plant to produce said plant.

36. The method according to claim 35, wherein said polynucleotide is an inhibitory polynucleotide.

37. The method according to claim 35, wherein said plant is modified in a DGAT trait.

38. The method according to claim 37, wherein said DGAT trait is chosen from TAG content, TAG composition, DGAT protein content and total protein content

39. The method according to claim 37, wherein said trait is a seed trait.

40. A plant produced by the method of claim 35.

41. Seeds produced by the plant of claim 40.

42. In a method of producing oil from seeds, the improvement comprising:
producing oil from said seeds of claim 41.

43. A method of producing a triacylglycerol, said method comprising:
contacting a diacylglycerol and fatty acid CoA with the plant DGAT polypeptide of claim 10 under conditions sufficient for said triacylglycerol to be produced.
